SEQUENCE LISTING (6 sequences)

SEQ ID NO: 1 - DNA, 621 bp, Zymomonas mobilis
ttaaggcgcgtaagccttgagaaaaagagaaacaatctgactgacatcggcctcgatttcttctttattcgcgagatttccttgggggatattataaagtctggaagctttactgcgcgcgcatagagagtgaagaaagaaagaggccgcatattcagggtcttcctgacggatctgtttcttttccatggctgaagcaaaataggatgccaatttccgcatggcggtttgaggccctgcttcgtaaaaaatttttccgacttcggggaaacgccaacattcaacaattgctagtcgaaacaaggctcttttatcaggttcgataatatttcgacagaaatgacagccatattggttcagcgcaatttcaatttcagcttgtggattaagcaaatctgtatagactttgtggaattcacccgccagccgttcaatcacagcccgaaacaaggcttcttttgaaggaaaatgagaccataacgtcccctttgatcctccgactttggccgcaatcgctgacatggaagtttcagcatatcctttttcaaggaaaaagcgttttgcctcttccagaatagcttctcggcgatcaatacctttatactgctttgatatattcat

SEQ ID NO: 2 - protein, 206 aa, Zymomonas mobilis
MNISKQYKGIDRREAILEEAKRFFLEKGYAETSMSAIAAKVGGSKGTLWSHFPSKEALFRAVIERLAGEFHKVYTDLLNPQAEIEIALNQYGCHFCRNIIEPDKRALFRLAIVECWRFPEVGKIFYEAGPQTAMRKLASYFASAMEKKQIRQEDPEYAASFFLHSLCARSKASRLYNIPQGNLANKEEIEADVSQIVSLFLKAYAP

SEQ ID NO: 3 - DNA, 2460 bp, Pseudomonas aeruginosa
acgagcgcggaaaaaccgtcagtttttttcccatcagatctgataggcattgcttatcattcgcgaatgcttagccgttgcagttgcgggtcggcgtcgatctgcctgtcgctgaacggcaaggtctgccattgctggcgggaaaacagctcggtctggtcgcggtagtgcggcgagcgcggatcgctggactgggagaaagccagcaacccgcgagccttgggcccttcctcggggaaggtcaccagctggatgtagctagtgccgccgaccacctccaggtggtcgcccttgcggacgctctggatcgcgttgtagaccccgaaatggccatcgccgccgggaatcgcgatgcgttcctggccacgggtgctcacttgcaggtcgccccagcgcgcgccgtcgggaatcccgctcttctccacctccgccgccgcgtccgccagcgcctggcgcacctgggtcgccacctgcggccggtcgagggcgatgccttgcggcgtatccaggggacgttgcgcatcgaacggttccttccacgcgccgtcgagttcggcgaagcgttgcatgaagcgctggaagtagacgaagccgctgccgctgtcgaggttggcgccacggtcccactgcgccagggccgcgcaggcgcgggcaagggacttctcgccctggttgtcgcggcacaggcggagcaggtccggcagcacctggtcggcgctgaagacatggttggcggtgaccatctcctcgagcgtcttcgcctccagcggctgcttgccctgtagccggctcagggcgtagcgcgcccgcggaccgatgggcttctcctggctgaccaggggcgagaagccctgcagcgggctcgccgggttggtcagccaggcgctgtcgttggagttctgcacgaagtccctgcgcaacagcaccggcagttgcgccgccggggtgatgccagcctgggccgcggccgggtcgcgactccaggcacagcggctgtcctgcccctggagggccggcaggccttcggcgaccagttgcggaatggcgcaggcgggaatcagttccggcttcaggtagggcaccaccgactggttcatgtacagggcgttgccctgctcgtccgcggccagggtgttgacccaggggatcccctgtagcgcctcgacgcgccggcgcaggtcggcgacgtcgctggcctggttgatcgagtaccactgttgcagtacccgggtgttctccaggttggcgtcacgcagcgcataggcctcgctgcggttccagtccagcttgccgggccagaccaccagcgggccgtagatcgactggtagaccttgtgctcgacgcgcgacagcttgccgtcggcgccgcgcacctcgatcgcgacggacttctcctccagcggcagcgaacgaccgtcgaccaggtagcgccgcgggtccttcgggtccagcgccaggcgatacagggtgaagtggctggaggtatccaccgtgtgggtccaggccaggtggcggctgaagccgatgttgaccaccggcaggccgggcagcgaggcccccatcacgtcgagccggccgggaatggtcaggtgcatctggtagaaacgcatcgcgccgttccaggggaagtgcgggttggccaggagcatgcccttgccgtccgccgaacgttcgctgccaacggcaatggcgttgctgccgcgctccaggcggaagcgctggcgccgctgctcggcgacctggaacgcctgctcgccgctcaaggcgaccttctccgctccgggcggcgcggcggccaccagcgcgtcggcgaactggccgaccccgccttcgaccagcaggcgccgggtcaggcgcagcaggtcatcggtcgcgatggcccgcagccagggctggccaaggcaactggtggtcttgccgtcggcctcgcggaggaagcggttgaaaccggcggcgtagccttcgagcaactggcgtaccgcgggcgtctgcgcctgccagaaggcttgcagcgcctcgggttggttgagccaggcgtagaagatgtcggacggcaggttgtccagctcggccgacgacttgccctcgctgccgaaatagcgcgcccgctcgccgcgcgcggtgacgatctcctcggccagcaggcaggcgttgtcgcgcgcgtaggcgtagccgatgccatagcccaggccgcgctcatccttggcccggatgtgcggcacgccataggcggtccagcggatatccgcggccagcccggtcggccgcggcatatcggcctggaccggcatcatcgaacccaacagcatgccggccaggccggtcagtacggtacgcatccccatcgatgtcgtttctctgcaaagtggtggccggaacggccgggacatgcaacgaaaacgccctgcgtgccgggcatcccctggcggggaaacgggcaacacagggcgtagcggcgtg

SEQ ID NO: 4 - protein, 762 aa, Pseudomonas aeruginosa
MGMRTVLTGLAGMLLGSMMPVQADMPRPTGLAADIRWTAYGVPHIRAKDERGLGYGIGYAYARDNACLLAEEIVTARGERARYFGSEGKSSAELDNLPSDIFYAWLNQPEALQAFWQAQTPAVRQLLEGYAAGFNRFLREADGKTTSCLGQPWLRAIATDDLLRLTRRLLVEGGVGQFADALVAAAPPGAEKVALSGEQAFQVAEQRRQRFRLERGSNAIAVGSERSADGKGMLLANPHFPWNGAMRFYQMHLTIPGRLDVMGASLPGLPVVNIGFSRHLAWTHTVDTSSHFTLYRLALDPKDPRRYLVDGRSLPLEEKSVAIEVRGADGKLSRVEHKVYQSIYGPLVVWPGKLDWNRSEAYALRDANLENTRVLQQWYSINQASDVADLRRRVEALQGIPWVNTLAADEQGNALYMNQSVVPYLKPELIPACAIPQLVAEGLPALQGQDSRCAWSRDPAAAQAGITPAAQLPVLLRRDFVQNSNDSAWLTNPASPLQGFSPLVSQEKPIGPRARYALSRLQGKQPLEAKTLEEMVTANHVFSADQVLPDLLRLCRDNQGEKSLARACAALAQWDRGANLDSGSGFVYFQRFMQRFAELDGAWKEPFDAQRPLDTPQGIALDRPQVATQVRQALADAAAEVEKSGIPDGARWGDLQVSTRGQERIAIPGGDGHFGVYNAIQSVRKGDHLEVVGGTSYIQLVTFPEEGPKARGLLAFSQSSDPRSPHYRDQTELFSRQQWQTLPFSDRQIDADPQLQRLSIRE

SEQ ID NO: 5 - DNA, 753 bp, Bacillus cereus
atgacagtaaaaaaactttatttcgttccagcaggtcgttgtatgttagatcattcttctgttaatagtacaatcgcgccgggaaatttattgaacttacctgtatggtgttatcttttggagacggaagaaggtcccattttagtagacacaggtatgccagaaagtgcagttaataatgaagggctttttaacggtacatttgttgaagggcagattttaccgaaaatgactgaagaagatagaatcgtaaatatattaaagcgtgtagggtatgagccggacgaccttttatatattattagttctcacttacattttgatcatgcaggaggaaacggtgcttttacaaatacaccgattattgtgcaacgaacggaatatgaggcagcacttcatagagaagaatatatgaaagaatgtatattaccgcatttgaactacaaaattattgaaggggattatgaagtggtaccaggtgttcaattattgtatacgccaggccattctccaggccatcagtcgctatccattgagacggagcaatccggttcaattttattgacgattgatgcatcttatacgaaagaaaattttgaagatgaagtaccgttcgcaggatttgatccagaattagctttatcttcaattaaacgtttaaaagaagttgtggcgaaagagaaaccaattattttctttggccatgatatagagcaggaaaagggttgtagagtgttccctgaatatatatag

SEQ ID NO: 6 - protein, 250 aa, Bacillus cereus
MTVKKLYFVPAGRCMLDHSSVNSTIAPGNLLNLPVWCYLLETEEGPILVDTGMPESAVNNEGLFNGTFVEGQILPKMTEEDRIVNILKRVGYEPDDLLYIISSHLHFDHAGGNGAFTNTPIIVQRTEYEAALHREEYMKECILPHLNYKIIEGDYEVVPGVQLLYTPGHSPGHQSLSIETEQSGSILLTIDASYTKENFEDEVPFAGFDPELALSSIKRLKEVVAKEKPIIFFGHDIEQEKGCRVFPEYI

(19) United States
(12) Patent Application Publication
(10) Pub. No.: US 2011/0124522 A1
(43) Pub. Date: May 26, 2011

(54) Methods of Disrupting Quorum Sensing to Affect Microbial Population Cell Density

(75) Inventors: Barry Marrs, Kennett Square, PA (US); Brian M. Swalla, Centreville, MD (US)

(73) Assignee: Athena Biotechnologies, Inc., Newark, DE (US)

(21) Appl. No.: 12/446,357

(22) PCT Filed: Oct. 26, 2007
(86) PCT No.: PCT/US07/22718

§ 371 Date: Feb. 9, 2011

Related U.S. Application Data
(60) Provisional application No. 60/854,874, filed on Oct. 27, 2006.

(51) Int. Cl. C40B 30/06; C12N 1/00; C12N 15/63; C12P 1/00; C12P 7/62; C12P 7/28; C12P 7/18; C12P 7/16; C12P 7/06

(52) U.S. Cl. 506/10; 435/243; 435/471; 435/41; 435/135; 435/150; 435/158; 435/160; 435/161

(57) ABSTRACT

The present invention relates to the modulation of quorum sensing mechanisms in a microorganism for the purpose of exploiting the fermentation capabilities of the microorganism.

### CROSS-REFERENCE TO RELATED APPLICATIONS

This is a National Stage application PCT International Application No. PCT/US2007/022718, filed Oct. 26, 2007, which in turn claims the benefit pursuant to 35 U.S. C.§119(e) of U.S. Provisional Application No. 60/854,874, filed on Oct. 27, 2006, which is hereby incorporated by reference in its entirety herein.

### BACKGROUND OF THE INVENTION

Several genera of bacteria have been shown to communicate with one another in order to coordinate the expression of specific genes in a cell density-dependent manner. This bacterial communication is called quorum sensing and it allows bacteria to control gene expression in response to the level of a diffusible signaling molecule called an autoinducer. Quorum sensing systems rely on the constitutive, low-level, expression of an autoinducer molecule that triggers the expression of a particular set of genes when its concentration in solution reaches some threshold level. This threshold is attained when a sufficient number (a “quorum”) of the bacteria are present in a localized area such that the combined rates of degradation and diffusional dilution of the autoinducer are less than its rate of production (See, e.g., U.S. Patent Application Publication No. 20040038374, which is hereby incorporated by reference herein in its entirety). Generally, the signaling molecule binds to a receptor protein, which then activates gene expression. Processes which have been described to be regulated by quorum sensing include virulence, bioluminescence, biofilm formation, swarming, sporulation, conjugal transfer of plasmids, and development of competence (Keller and Surette, 2006, Nat. Rev. Microbiol., 4:249-258; Milton, 2006, Int. J. Med. Microbiol., 296:61-71; Walters and Sperandio, 2006, Int. J. Med. Microbiol., 296:125-31).

Bacteria differ in the type of autoinducer produced. It appears that Gram-negative bacteria typically produce acyl homoserine lactones (AHL) and Gram-positive bacteria typically produce peptides, but differences also occur within these groupings. For example, a given Gram-negative bacterium may produce one or multiple acyl lactones (ACLs), including N-(3-oxohexanoyl)-L-homoserine lactone (OHHL), N-(3-oxododecanoyl)-L-homo-serine lactone (OdDHL), N-butanoyl-L-homoserine lactone (BHL), and N-hexanoyl-L-homoserine lactone (HHL). These differences in the acyl chain affect the biological properties of the autoinducers, and allow for specificity to a particular bacterial genotype or group, genetic control based on interacting AHL, and autoinducer crosstalk and interferences among bacterial genotypes (Swift et al., 1996, Trends Microbiol., 4:463-465).

Three main types of quorum sensing systems have thus far been described in bacteria: Type 1, Type 2 and peptide-based. Type 1 quorum sensing has so far only been demonstrated in Gram-negative microorganisms and utilizes acyl homoserine lactones as signaling molecules. Type 2 has been demonstrated in both Gram positive and Gram negative microorganisms and is believed to utilize 4-hydroxy-5-methyl-2H-furan-3-one or 4,5-dihydroxy-2-cyclopenten-l-one as the signaling molecule. Peptide-based quorum sensing systems have been demonstrated only in Gram positive microorganisms and rely on short peptides for gene activation. In addition, other chemical signals have been shown to be used for quorum sensing; these include gamma butyrolactone in Streptomyces sp. and 2-heptyl-3-hydroxy-4-quinolone in Pseudomonas aeruginosa.

Type 1 quorum sensing utilizes acyl homoserine lactones (AHL) as signaling molecules. AHL chemical signals consist of a lactone ring attached to an acyl chain by means of a peptide bond. The acyl chain length and modification varies with the species of microorganism or the process regulated. Some AHL contain a carbonyl or hydroxyl group at the 3 position of the acyl chain (e.g., 3-oxo-hexanoyl homoserine lactone and 3-hydroxy-butanoyl homoserine lactone). The best-characterized Type 1 quorum sensing system is the Vibrio fischeri luxI/luxR system (Kaplan and Greenberg, 1985, J. Bacteriol., 163:1210-1214). It consists of two genes, luxI and luxR. The expression of luxI and luxR is responsible for the production and detection of the autoinducer. The luxI protein catalyzes the synthesis of the autoinducer 3-oxo-hexanoyl homoserine lactone (OHHL). As the cell density increases the autoinducer accumulates and when a threshold level is reached, the OHHL signal interacts with the luxR protein. The luxR/OHHL complex binds to DNA at the lux box resulting in transcription of the bioluminescence genes.

Other microorganisms exhibiting Type 1 quorum sensing possess analogs of luxI and luxR and subsequent research has revealed the presence of genes homologous to luxI and luxR in many other bacteria which regulate genes involved in numerous other microbial processes. Proteins homologous to the LuxR family of auto-inducer dependent transcriptional activator proteins are found across a wide array of different bacterial species. Two well characterized examples include the archetype LuxR protein from Vibrio fischeri and the TraR protein of Agrobacter tumefaciens, which regulate the expression of genes required for light production or conjugal plasmid transfer, respectively, in response to the concentration of specific extracellular AHL signaling molecules.

International patent application WO 01/85664 is incorporated herein in its entirety for its description of Type 2 quorum sensing. Biosynthesis of the Type 2 autoinducer is believed to proceed through progressive steps from methionine through S-adenosyl methionine to S-adenosyl homocysteine to S-ribosyl homocysteine to 4-hydroxy-5-methyl-2H-furan-3-one or 4,5-dihydroxy-2-cyclopenten-1-one. Enzymes involved in the synthesis are believed to include methionine adenosyl transferase, methyl transferase, nucleosidase and the luxS protein or its analogs, which synthesizes 4-hydroxy-5-methyl-2H-furan-3-one or 4,5- dihydroxy-2-cyclopenten-1-one from its precursor. In Vibrio harveyi, the receptors for the Type 2 autoinducer are luxP and luxPQ. When autoinducer concentrations reach a threshold level, the autoinducer interacts with the receptor and luxO is dephosphorylated (and inactivated), thereby preventing activation of a repressor and allowing luxR to activate transcription of the luxCDABE genes.

Many Gram positive bacteria use secreted peptides as autoinducers. Generally, in peptide based quorum sensing systems, the peptide is secreted by an ATP-binding cassette (ABC) transporter. The concentration of the autoinducer increases with cell density, and at a threshold level two component sensor kinases detect the autoinducer. A phoshorylation cascade is initiated which results in phosphorylation of a cognate response regulator protein. The response regulator is thus activated, allowing it to bind DNA and affect transcription of the quorum-sensing regulated genes.

It has been demonstrated that enzymes can degrade AHL. Lactonase has been shown to inactivate oxohexanoyl-, oxodecanoyl- and oxooctanoyl-homoserine lactones (Dong et al., 2000, PNAS, 97:3526-331; Dong et al., 2001, Nature 411:813-817). Certain organisms, including several species of bacteria, are known to produce two types of enzymes that degrade AHL signal compounds through two different reaction mechanisms. AHL-lactonase enzymes degrade AHL molecules by hydrolyzing the lactone bond to produce acyl-homoserine, and AHL-acylases cleave the amide bond of AHL molecules to separate the acyl and homoserine lactone moieties. For example, Bacillus cereus and Agrobacterium tumefaciens produce the AHL-lactonase enzymes AiiA and AttM, respectively, and Ralstonia and Pseudomonas aeruginosa produce the

AHL-acylases AiiD and PvdQ, respectively. Similarly, it has been demonstrated that a strain of Variovorax paradoxus can utilize several acyl homoserine lactones for growth; it is believed that the ring is enzymatically cleaved allowing the acyl chain and lactone ring to be used as sources of energy and nitrogen, respectively (Leadbetter and Greenberg, 2000, J. Bacteriology, 182:6921-6926).

Microorganisms produce a diverse array of fermentation products. These products include organic acids, such as lactate, acetate, succinate and butyrate, as well as neutral products such as ethanol, butanol, acetone and butanediol. Indeed, the diversity of fermentation products from bacteria has led to their use as a primary determinant in taxonomy. See, for example, Bergey's Manual of Systematic Bacteriology, Williams & Wilkins Co., Baltimore (1984). The microbial production of these fermentation products, by a variety of fermentation culture methods including, adhered or suspended, and batch or continuous, forms the basis of many economically successful applications of biotechnology, including the production of dairy products, meats, beverages and fuels. In recent years, many advances have been made in the field of biotechnology as a result of new technologies which enable researchers to selectively modify the genetic makeup of some microorganisms.

Z. mobilis is an obligatively fermentative bacterium which lacks a functional system for oxidative phosphorylation. Like the yeast Saccharomyces cerevisiae, Z. mobilis produces ethanol and carbon dioxide as principal fermentation products. Z. mobilis produces ethanol by a short pathway which requires only two enzymatic activities: pyruvate decarboxylase and alcohol dehydrogenase. Pyruvate decarboxylase is the key enzyme in this pathway which diverts the flow of pyruvate to ethanol. Pyruvate decarboxylase catalyzes the nonoxidative decarboxylation of pyruvate to produce acetaldehyde and carbon dioxide. Two alcohol dehydrogenase isozymes are present in this organism and catalyze the reduction of acetaldehyde to ethanol during fermentation, accompanied by the oxidation of NADH to NAD+. Although bacterial alcohol dehydrogenases are common in many organisms, few bacteria have pyruvate decarboxylase. Attempts to modify Z. mobilis to enhance its commercial utility as an ethanol producer have met with very limited success.

Genetic-engineering approaches, for example, for the addition of saccharifying traits to microorganisms for the production of ethanol or lactic acid have been directed at the secretion of high enzyme levels into the medium. That is, the art has also concerned itself with modifying microorganisms already possessing the requisite proteins for transporting cellularly-produced enzymes into the fermentation medium, where those enzymes can then act on the polysaccharide substrate to yield mono- and oligosaccharides. This approach has been taken because the art has perceived difficulty in successfully modifying organisms lacking the requisite ability to transport such proteins.

The genes encoding alcohol dehydrogenase II and pyruvate decarboxylase in Z. mobilis have been separately cloned, characterized, and expressed in E. coli. See Brau & Sahm (1986a) Arch. Microbiol. 144:296-301, (1986b) Arch. Microbiol. 146:105-110; Conway et al. (1987a) J. Bacteriol. 169:2591-2597; Neale et al. (1987) Nucleic Acids Res. 15:1752-1761; Ingram and Conway [1988] Appl. Environ. Microbiol. 54:397-404; Ingram et al. (1987) Appl. Environ. Microbiol. 53:2420-2425.

Brau and Sahm (1986a), supra, first demonstrated that ethanol production could be increased in recombinant E. coli by the over-expression of Z. mobilis pyruvate decarboxylase although very low ethanol concentrations were produced. Subsequent studies extended this work by using two other enteric bacteria, Erwinia chrysanthemi and Klebsiella planticola, and thereby achieved higher levels of ethanol from hexoses, pentoses, and sugar mixtures. See Tolan and Finn (1987) Appl. Environ. Microbiol. 53:2033-2038, 2039-2044. The genes encoding pyruvate decarboxylase (pdc) and alcohol dehydrogenase II (adhB) from Zymomonas mobilis have been expressed at high levels in Gram-negative bacteria, effectively redirecting fermentative metabolism to produce ethanol as the primary product (Beall et al., 1993; Ingram and Conway, 1988; Wood and Ingram, 1992).

Suitable microorganisms capable of growing to sufficient density to allow for high-yield production of fermentation products, including ethanol, have been sought for many years. Quorum sensing may be involved in limiting the population cell density. Such a mechanism of maintaining a limited cell density may contribute to the difficulties experienced by those who have tried to establish increased-density cultures of some bacteria. In applications where production yield can be increased through an increase in population cell density or volumetric productivity, disruption of the quorum sensing systems of microbial populations should lead to an increase in yield. The present invention addresses and solves this problem.

It is known that some microorganisms utilize quorum sensing to control their cell division, and thus many microorganisms have been uncultivable in the laboratory due to quorum sensing. Thus, there is a long felt need to identify and discover ways to uncover potential novel genes and microorganism from a sample where the microorganisms have previously been “uncultivated”. The present invention satisfies this need.

### BRIEF SUMMARY OF THE INVENTION

The invention includes a genetically modified known microorganism comprising at least one genetic mutation, wherein the mutation confers upon the genetically modified microorganism the ability to grow to a greater cell density than the cell density of an otherwise identical microorganism that does not comprise the mutation and is cultured under identical culture conditions. Preferably, the mutation is a deletion mutant.

In one embodiment, the genetically modified known microorganism comprises a mutation within the regulatory region of a gene associated with quorum sensing.

In another embodiment, the genetically modified known microorganism comprises a mutation in a nucleic acid sequence encoding a quorum sensing protein; wherein the mutation modulates at least one of:
a. the production of the quorum sensing protein;
b. the half-life of the quorum sensing protein;
c. the response of the quorum sensing protein to a quorum sensing signal;
d. the activity of the quorum sensing protein; and
e. the interaction of the quorum sensing protein with a quorum sensing pathway in the microorganism.

In yet another embodiment, the genetically modified known microorganism comprises a mutation that modulates production and/or activity of at least one polypeptide involved in quorum sensing signaling in at least one pathway selected from the group consisting of a Type 1 quorum sensing pathway, a Type 2 quorum sensing pathway, and a peptide-mediated quorum sensing pathway.

In another embodiment, the genetically modified known microorganism comprises a mutation that is a transposable interruptor resulting in interruption of the nucleic acid encoding a polypeptide involved in quorum sensing signaling in at least one pathway selected from the group consisting of a Type 1 quorum sensing pathway, a Type 2 quorum sensing pathway, and a peptide-mediated quorum sensing pathway.

In another embodiment, the genetically modified known microorganism comprises a mutation in a nucleic acid sequence encoding a LuxR-type protein; wherein the mutation modulates at least one of:
a. the binding of the LuxR-type protein to DNA;
b. the binding of the LuxR-type protein to an acyl homoserine lactone (AHL); and
c. the protein folding switch of the LuxR-type protein. conditions.

The invention also includes a genetically modified known microorganism comprising at least one genetic, wherein the mutation confers upon the genetically modified microorganism the ability to achieve a higher volumetric productivity for a fermentation product produced by the microorganism than the volumetric productivity for the same fermentation product by an otherwise identical microorganism that does not comprise the mutation.

The invention includes a method of increasing the cell density of a population of known microorganisms comprising:
a. introducing a genetic modification into a microorganism; and
b. growing the genetically modified microorganism in a culture medium, whereby the modified microorganism grows to a greater cell density than the cell density of an otherwise identical microorganism that does not comprise the mutation and is cultured under identical culture conditions.

The invention includes a method of increasing the volumetric productivity of a population of known microorganisms comprising:
a. introducing a genetic modification into a microorganism; and
b. growing the modified microorganism in a culture medium,
   wherein the volumetric productivity of the modified microorganism with respect to a fermentation product produced by the microorganism is greater than the volumetric productivity for the same fermentation product by an otherwise identical microorganism that does not comprise the mutation.

The invention includes a method of increasing the cell density of a population of known microorganism comprising:
a. introducing into a microorganism a nucleic acid vector comprising a nucleic acid sequence encoding a polypeptide, wherein the polypeptide has the ability to modulate at least one quorum sensing pathway;
b. expressing the polypeptide within the microorganism; and
c. growing the modified microorganism in a culture medium,
   whereby the modified microorganism grows to a greater cell density than the cell density of an otherwise identical microorganism that does not comprise the polypeptide and is cultured under identical culture conditions.

The invention includes a method of increasing the volumetric productivity of a population of known microorganism comprising:
a. introducing into a microorganism a nucleic acid vector comprising a nucleic acid sequence encoding a polypeptide, wherein the polypeptide has the ability to modulate at least one quorum sensing pathway;
b. expressing the polypeptide within the microorganism; and
c. growing the modified microorganism in a culture medium,
   wherein the volumetric productivity of the modified microorganism with respect to a fermentation product produced by the microorganism is greater than the volumetric productivity for the same fermentation product by an otherwise identical microorganism that does not comprise the polypeptide.

The invention includes a method of producing a fermentation product comprising:
a. providing a genetically modified known microorganism comprising at least one mutation in a nucleic acid sequence encoding a quorum sensing protein; wherein the mutation modulates at least one of:

i. the production of the quorum sensing protein;

ii. the half-life of the quorum sensing protein;

iii. the response of the quorum sensing protein to a quorum sensing signal;

iv. the activity of the quorum sensing protein; and

v. the interaction of the quorum sensing protein with a quorum sensing pathway in the microorganism; and
b. culturing the genetically modified microorganism in a culture medium; wherein the mutation confers upon the genetically modified microorganism the ability to achieve a higher volumetric productivity for a fermentation product produced by the microorganism than the volumetric productivity for the same fermentation product by an otherwise identical microorganism that does not comprise the mutation.

The invention includes a method of producing a fermentation product comprising:
a. introducing into a known microorganism a nucleic acid vector comprising a nucleic acid sequence encoding a polypeptide, wherein the polypeptide has the ability to modulate at least one quorum sensing pathway; and
b. culturing the genetically modified microorganism in a culture medium; wherein the modified microorganism has the ability to achieve a higher volumetric productivity for a fermentation product produced by the microorganism than the volumetric productivity for the same fermentation product by an otherwise identical microorganism that does not comprise the polypeptide.

The invention includes a method of identifying a gene associated with quorum sensing, wherein mutation of the gene in a microbial cell allows the cell to grow at an increased density. The method comprises:
a. introducing a library of mutant nucleic acid fragments into a plurality of cells;
b. selecting a cell exhibiting increased cell growth;
c. isolating the mutated nucleic acid sequence from the cell exhibiting increased cell growth;
d. sequencing the mutated nucleic acid;
e. analyzing the sequence of the mutated nucleic acid sequence; thereby identifying a gene associated with quorum sensing.

### BRIEF DESCRIPTION OF THE DRAWINGS

For the purpose of illustrating the invention, there are depicted in the drawings certain embodiments of the invention. However, the invention is not limited to the precise arrangements and instrumentalities of the embodiments depicted in the drawings.

FIG. 1 is an image of a pAB301 plasmid according to the present invention.

FIG. 2 is an image of a pAB303 plasmid according to the present invention.

FIG. 3, comprising FIGS. 3A and 3B is a schematic of transposon insertion mutagenesis. FIG. 3A is a cartoon showing in vitro assembly of the Mu transpososome and synthetic DNA to produce a transpososome. FIG. 3B is a schematic diagram of mini-Mu DNA structure.

### DETAILED DESCRIPTION OF THE INVENTION

Some bacteria produce chemical signals that regulate their own cell density. It has been suggested that quorum sensing signal molecules may inhibit the growth of daughter cells of the bacteria producing the quorum sensing signal molecules thereby poising the cell population at low density. Such a mechanism of sustaining a relatively low cell density may also contribute to the difficulties experienced by microbiologists in trying to establish pure cultures of these bacteria. Removal of the signal, blocking its production or inhibiting the activity of the signal by way of disrupting a component of a quorum sensing system may allow cell density to increase and thereby allow for the cultivation of the cell that previously was difficult to grow or was even uncultivatable.

The invention relates to methods and compositions for mutating a gene in a known microorganism, whereby mutation of the gene increases the ability of the microorganism to be cultivated compared to an otherwise identical microorganism e wherein the same gene is not mutated. In some instances, the gene that is mutated is a gene that is essential for limiting cell density (e.g., quorum sensing system). Accordingly, the invention encompasses screening and identifying genes associated with limiting cell density and mutating such genes in a microorganism to enhance the ability of the microorganism to grow at a higher density where otherwise the microorganism would not grow at all, or grow to a lower cell density.

The present invention also relates to methods and compositions for increasing microbial population cell density by disruption of a quorum sensing system that limits microbial population cell density. By modulating one or more quorum sensing systems in a known microorganism according to the present invention, referred to herein in one embodiment as “quorum sensing quenching,” increased yields of fermentation products can be obtained. One such product is ethanol. By way of a non-limiting example, the yield of ethanol obtained from a culture of a microorganism can be increased according to the methods of the present invention, wherein one or more quorum sensing systems in the microorganism is disrupted, thereby increasing the cell density to which the microorganism can grow.

### Definitions

The articles “a” and “an” are used herein to refer to one or to more than one (i.e. to at least one) of the grammatical object of the article. By way of example, “an element” means one element or more than one element.

As used herein, the phrase “acyl homoserine lactone-degrading enzyme” or “AHL-degrading enzyme” is an enzyme that catalyzes the modification and/or breakdown of an acyl homoserine lactone. In one aspect, an AHL-degrading enzyme degrades an acyl homoserine lactone by adding one or more atoms to the acyl homoserine lactone. In another aspect, an AHL-degrading enzyme degrades an acyl homoserine lactone by breaking one or more bonds in the acyl homoserine lactone. In yet another aspect, an AHL-degrading enzyme degrades an acyl homoserine lactone by removing one or more atoms from the acyl homoserine lactone.

The term “antibody,” as used herein, refers to an immunoglobulin molecule which is able to specifically bind to a specific epitope on an antigen. Antibodies can be intact immunoglobulins derived from natural sources or from recombinant sources and can be immunoreactive portions of intact immunoglobulins. Antibodies are typically tetramers of immunoglobulin molecules. The antibodies in the present invention may exist in a variety of forms including, for example, polyclonal antibodies, monoclonal antibodies, Fv, Fab and F(ab)₂, as well as single chain antibodies and humanized antibodies (Harlow et al., 1999, Using Antibodies: A Laboratory Manual, Cold Spring Harbor Laboratory Press, NY; Harlow et al., 1989, Antibodies: A Laboratory Manual, Cold Spring Harbor, New York; Houston et al., 1988, Proc. Natl. Acad. Sci. USA 85:5879-5883; Bird et al., 1988, Science 242:423-426).

The term “augment” is used herein to indicate an increase in the quantity or quality of something. By way of several non-limiting examples, the production of a polypeptide is “augmented” if any amount of polypeptide is produced, when there previously was no polypeptide produced in a cell. The production of a polypeptide is “augmented” if any amount of polypeptide is produced, when there previously was no measurable polypeptide existing within a cell. The production of a polypeptide is also “augmented” according to the invention if an increased amount of polypeptide is produced in a cell, when there previously was a lesser level of polypeptide existing within a cell.

As used herein, the term “biochemical pathway” refers to a connected series of biochemical reactions normally occurring in a cell, or more broadly a cellular event such as cellular division or DNA replication. Typically, the steps in such a biochemical pathway act in a coordinated fashion to produce a specific product or products or to produce some other particular biochemical action. Such a biochemical pathway requires the expression product of a gene if the absence of that expression product either directly or indirectly prevents the completion of one or more steps in that pathway, thereby preventing or significantly reducing the production of one or more normal products or effects of that pathway.

A “conservative substitution” is the substitution of an amino acid with another amino acid with similar physical and chemical properties. In contrast, a “nonconservative substitution” is the substitution of an amino acid with another amino acid with dissimilar physical and chemical properties.

As used herein, the terms “gene” and “recombinant gene” refer to nucleic acid molecules comprising an open reading frame encoding a polypeptide.

As used herein, the term “genetically engineered” refers to a modification of the inherent genetic material of a microorganism (e.g., one or more of the deletion, addition, or mutation of one or more nucleic acid residues within the genetic material), additional of exogenous genetic material to a microorganism (e.g., stable plasmid, integrating plasmid, naked genetic material, among other things), causing the microorganism to alter its genetic makeup due to external or internal signaling (e.g., environmental pressures, chemical pressures, among other things), or any combination of these or similar techniques for altering the overall genetic makeup of the organism.

As used herein, “homology” is used synonymously with “identity.”

The determination of percent identity between two nucleotide or amino acid sequences can be accomplished using a mathematical algorithm. For example, a mathematical algorithm useful for comparing two sequences is the algorithm of Karlin and Altschul (1990, Proc. Natl. Acad. Sci. USA 87:2264-2268), modified as in Karlin and Altschul (1993, Proc. Natl. Acad. Sci. USA 90:5873-5877). This algorithm is incorporated into the NBLAST and XBLAST programs of Altschul, et al. (1990, J. Mol. Biol. 215:403-410), and can be accessed, for example at the National Center for Biotechnology Information (NCBI) world wide web site having the universal resource locator “http://www.ncbi.nlm.nih.gov/BLAST/”. BLAST nucleotide searches can be performed with the NBLAST program (designated “blastn” at the NCBI web site), using the following parameters: gap penalty=5; gap extension penalty=2; mismatch penalty=3; match reward=1; expectation value 10.0; and word size=11 to obtain nucleotide sequences homologous to a nucleic acid described herein. BLAST protein searches can be performed with the XBLAST program (designated “blastn” at the NCBI web site) or the NCBI “blastp” program, using the following parameters: expectation value 10.0, BLOSLTM62 scoring matrix to obtain amino acid sequences homologous to a protein molecule described herein. To obtain gapped alignments for comparison purposes, Gapped BLAST can be utilized as described in Altschul et al. (1997, Nucleic Acids Res. 25:3389-3402). Alternatively, PSI-Blast or PHI-Blast can be used to perform an iterated search which detects distant relationships between molecules (Id.) and relationships between molecules which share a common pattern. When utilizing BLAST, Gapped BLAST, PSI-Blast, and PHI-Blast programs, the default parameters of the respective programs (e.g., XBLAST and NBLAST) can be used. See http://www.ncbi.nlm.nih.gov/BLAST/. The percent identity between two sequences can be determined using techniques similar to those described above, with or without allowing gaps. In calculating percent identity, typically exact matches are counted.

“Homologous” as used herein, refers to the subunit sequence similarity between two polymeric molecules, e.g., between two nucleic acid molecules, e.g., two DNA molecules or two RNA molecules, or between two polypeptide molecules. When a subunit position in both of the two molecules is occupied by the same monomeric subunit, e.g., if a position in each of two DNA molecules is occupied by adenine, then they are homologous at that position. A first region is homologous to a second region if at least one nucleotide residue position of each region is occupied by the same residue. Homology between two regions is expressed in terms of the proportion of nucleotide residue positions of the two regions that are occupied by the same nucleotide residue. The homology between two sequences is a direct function of the number of matching or homologous positions, e.g., if half (e.g., five positions in a polymer ten subunits in length) of the positions in two compound sequences are homologous then the two sequences are 50% homologous, if 90% of the positions, e.g., 9 of 10, are matched or homologous, the two sequences share 90% homology. By way of example, the DNA sequences 3′ATTGCC5′ and 3′TATGGC share 50% homology.

An “isolated nucleic acid” refers to a nucleic acid segment or fragment which has been separated from sequences which flank it in a naturally occurring state, e.g., a DNA fragment which has been removed from the sequences which are normally adjacent to the fragment, e.g., the sequences adjacent to the fragment in a genome in which it naturally occurs. The term also applies to nucleic acids which have been substantially purified from other components which naturally accompany the nucleic acid, e.g., RNA or DNA or proteins, which naturally accompany it in the cell. The term therefore includes, for example, a recombinant DNA which is incorporated into a vector, into an autonomously replicating plasmid or virus, or into the genomic DNA, or which exists as a separate molecule (e.g., as a cDNA or a genomic or cDNA fragment produced by PCR or restriction enzyme digestion) independent of other sequences. It also includes a recombinant DNA which is part of a hybrid gene encoding additional polypeptide sequence.

A “polynucleotide” means a single strand or parallel and anti-parallel strands of a nucleic acid. Thus, a polynucleotide may be either a single-stranded or a double-stranded nucleic acid.

The term “nucleic acid” typically refers to a large polynucleotide.

The term “oligonucleotide” typically refers to short a polynucleotide, generally, no greater than about 50 nucleotides. It will be understood that when a nucleotide sequence is represented by a DNA sequence (i.e., A, T, G, C), this also includes an RNA sequence (i.e., A, U, G, C) in which “U” replaces “T.”

Conventional notation is used herein to describe polynucleotide sequences: the left-hand end of a single-stranded polynucleotide sequence is the 5′-end; the left-hand direction of a double-stranded polynucleotide sequence is referred to as the 5′-direction.

The direction of 5′ to 3′ addition of nucleotides to nascent RNA transcripts is referred to as the transcription direction. The DNA strand having the same sequence as an mRNA is referred to as the “coding strand”; sequences on the DNA strand which are located 5′ to a reference point on the DNA are referred to as “upstream sequences”; sequences on the DNA strand which are 3′ to a reference point on the DNA are referred to as “downstream sequences.”

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

Unless otherwise specified, a “nucleotide sequence encoding an amino acid sequence” includes all nucleotide sequences that are degenerate versions of each other and that encode the same amino acid sequence. Nucleotide sequences that encode proteins and RNA may include introns.

In the context of the present invention, the following abbreviations for the commonly occurring nucleic acid bases are used. “A” refers to adenosine, “C” refers to cytidine, “G” refers to guanosine, “T” refers to thymidine, and “U” refers to uridine.

“Recombinant polynucleotide” refers to a polynucleotide having sequences that are not naturally joined together. An amplified or assembled recombinant polynucleotide may be included in a suitable vector, and the vector can be used to transform a suitable host cell. A recombinant polynucleotide may serve a non-coding function (e.g., promoter, enhancer, origin of replication, ribosome-binding site, etc.) as well.

A “recombinant polypeptide” is one which is produced upon expression of a recombinant polynucleotide.

As used herein, the term “promoter/regulatory sequence” means a nucleic acid sequence which is required for expression of a gene product operably linked to the promoter/regulator sequence. In some instances, this sequence may be the core promoter sequence and in other instances, this sequence may also include an enhancer sequence and other regulatory elements which are required for expression of the gene product. The promoter/regulatory sequence may, for example, be one which expresses the gene product in a condition-specific manner.

As used herein, the term “protein folding switch,” refers to a change in the conformation of a polypeptide, or a portion of a polypeptide, in which the change in conformation modulates a physical, chemical or biological activity.

“Mutants,” “derivatives,” and “variants” of a polypeptide (or of the DNA encoding the same) are polypeptides which may be modified or altered in one or more amino acids (or in one or more nucleotides) such that the peptide (or the nucleic acid) is not identical to the wild-type sequence, but has homology to the wild type polypeptide (or the nucleic acid).

A “mutation” of a polypeptide (or of the DNA encoding the same) is a modification or alteration of one or more amino acids (or in one or more nucleotides) such that the peptide (or nucleic acid) is not identical to the sequences recited herein, but has homology to the wild type polypeptide (or the nucleic acid).

As used herein, a “mutant form” of a gene is a gene which has been altered, either naturally or artificially, changing the base sequence of the gene, which results in a change in the amino acid sequence of an encoded polypeptide. The change in the base sequence may be of several different types, including changes of one or more bases for different bases, small deletions, and small insertions. Mutations may also include transposon insertions that lead to attenuated activity, i.e., by resulting in expression of a truncated protein. By contrast, a normal form of a gene is a form commonly found in a natural population of an organism. Commonly a single form of a gene will predominate in natural populations. In general, such a gene is suitable as a normal form of a gene; however, other forms which provide similar functional characteristics may also be used as a normal gene.

“Polypeptide” refers to a polymer composed of amino acid residues, related naturally occurring structural variants, and synthetic non-naturally occurring analogs thereof linked via peptide bonds, related naturally occurring structural variants, and synthetic non-naturally occurring analogs thereof. Synthetic polypeptides can be synthesized, for example, using an automated polypeptide synthesizer.

The term “protein” typically refers to large polypeptides.

The term “peptide” typically refers to short polypeptides.

Conventional notation is used herein to portray polypeptide sequences: the left-hand end of a polypeptide sequence is the amino-terminus; the right-hand end of a polypeptide sequence is the carboxyl-terminus.

A “portion” of a polynucleotide means at least about twenty sequential nucleotide residues of the polynucleotide. It is understood that a portion of a polynucleotide may include every nucleotide residue of the polynucleotide.

The term “known” as it refers to a microorganism means a microorganism, preferably a bacterium, that has been identified prior to genetic manipulation to alter quorum sensing as described herein. Such identification includes, at the least, isolation of the organism and optionally culturing of the microorganism such that the stated genetic manipulation can be conducted.

The term “modulate,” as used herein, refers to any change from the present state. The change may be an increase or a decrease. For example, the activity of an enzyme may be modulated such that the activity of the enzyme is increased from its current state. Alternatively, the activity of an enzyme may be modulated such that the activity of the enzyme is decreased from its current state.

As the term is used herein, “population” refers to two or more cells.

As used herein, the term “quorum sensing quenching” refers to the interference with, disruption of, or inhibition of at least one quorum sensing pathway in a microorganism.

The term “library” designates a complex composition comprising a plurality of polynucleotides, of various origins and structure. Typically, some polynucleotides within the library are unknown polynucleotides, i.e., of polynucleotides whose sequence and/or source and/or activity is not known or characterized. In addition to such unknown (or uncharacterized) polynucleotides, the library may further include known sequences or polynucleotides. Typically, the library comprises more than 20 distinct polynucleotides, more preferably at least 50, typically at least 100, 500 or 1000. The complexity of the libraries may vary. In particular, libraries may contain more than 5000, 10 000 or 100 000 polynucleotides, of various origin, source, size, etc. Furthermore, the polynucleotides are generally cloned into cloning vectors, allowing their maintenance and propagation in suitable host cells. The polynucleotides in the library may be in the form of a mixture or separated from each other, in all or in part. It should be understood that some or each polynucleotide in the library may be present in various copy numbers. Examples of a type of library include, but is not limited to, a gene disruption library or otherwise a mutant insertional library, genomic library, cDNA screening library, and the like. Furthermore, a type of gene disruption library includes, but is not limited to a signature-tagged mutant library, a transposon insertion mutant library, and the like. In addition to nucleic acid libraries, analogous libraries of polypeptides are also contemplated.

With respect to a library of transposon insertion sites, the library is a collection of sequence information, which information is provided in either biochemical form (e.g., as a collection of polynucleotide molecules), or in electronic form (e.g., as a collection of polynucleotide sequences stored in a computer-readable form, as in a computer system and/or as part of a computer program). The sequence information of the polynucleotides can be used in a variety of ways, for instance as a resource for gene discovery, i.e., for identifying and verifying genes associated with quorum sensing, or for identifying essential or important homologues in other biochemical pathways. A polynucleotide sequence in a library can be a polynucleotide that represents an mRNA, polypeptide, or other gene product encoded by the polynucleotide, and accordingly such a polynucleotide library could be used to formulate corresponding RNA or amino acid libraries according to the sequences of the library members. Biochemical embodiments of the library include a collection of nucleic acids that have the sequences of the genes or transposon insertion sites in the library, where the nucleic acids can correspond to the entire gene in the library or to a fragment thereof

As used interchangeably herein, the terms “transposon” and “transposable element” are intended to include a piece of DNA that can insert into and cut itself out of, genomic DNA of a particular host species. Transposons can include mobile genetic elements (MGEs) containing insertion sequences and additional genetic sequences unrelated to insertion functions (for example, sequences encoding a reporter gene).

As used herein, the term “volumetric productivity” refers to the amount of a particular product obtained in a particular unit volume within a particular unit of time. By way of a non-limiting example, the volumetric productivity of a bacterial cell culture can be measured for the amount of a fermentation product produced per milliliter of culture per minute.

### Description

### I. Genetic Modification

The invention features a genetically modified microorganism comprising at least one mutation in a gene encoding a protein involved in limiting cell density in the microorganism (e.g., a quorum sensing protein). The microorganism is an already known microorganism. In some instances, the mutation modulates at least one of the production of the quorum sensing protein, the half-life of the quorum sensing protein, the biological activity of the quorum sensing protein, the response of the quorum sensing protein to a quorum sensing signal, and the interaction of the quorum sensing protein with a quorum sensing pathway in the microorganism. In some instances, the mutation can be within the regulatory sequence of the gene (e.g., promoter sequence). Regardless, the mutation confers upon the genetically modified microorganism the ability to achieve a higher volumetric productivity for a fermentation product produced by the microorganism than exists in the absence of the mutation.

The mutation can also occur in genes that regulate quorum sensing proteins. The invention features a genetically modified microorganism comprising at least one mutation in a gene encoding a quorum sensing protein, wherein the mutation modulates at least one of the production of the quorum sensing protein, the half-life of the quorum sensing protein, the response of the quorum sensing protein to a quorum sensing signal, and the interaction of the quorum sensing protein with a quorum sensing pathway in the microorganism. The mutation confers upon the genetically modified microorganism the ability to achieve a higher volumetric productivity for a fermentation product produced by the microorganism than exists in the absence of the mutation.

The present invention differs from methods in the prior art, because the present invention is directed to an element of endogenous control of the quorum sensing pathway of a known microorganism. The prior art methods disclose solely exogenous control of such quorum-sensing pathways. For example, U.S. Patent Application Publication No. 20040038374 of Kuhner, et al., discloses the exogenous addition of an agent to affect a quorum sensing pathway in a microorganism. Such prior art methods have limitations and drawbacks, including varied ability of such agents to enter the microorganism, the availability of such agents when in the culture medium, and the stability of such agents in culture medium.

In another aspect of the present invention, the mutation confers upon the genetically modified microorganism the ability to grow to a greater cell density than in the absence of the mutation. It will be understood, based on the disclosure set forth herein, that the volumetric productivity and the cell density of a microorganism culture may or may not be related, based on parameters as described in detail elsewhere herein.

The present invention features methods and compositions for quorum sensing quenching by way of one or more mechanisms, at least one of which involves endogenous control in a microorganism. As will be understood based on the disclosure set forth herein, any quorum sensing pathway in the microorganism can be a target according to the present invention. By way of a non-limiting example, both AHL-lactonase and AHL-acylase enzymes degrade AHL molecules into products that are not recognized by the bacteria as quorum-sensing signal molecules (Huang 2003, App Env Micro 69:5941-5949; Zhang et al. 2002 PNAS 99:4638-4643). Heterologous expression of a cloned AHL-degrading enzyme can thus alter the response of a bacterial strain to endogenously produced AHL signal molecules (Lin et al 2003 Mol Microbiol 47:849-860).

Novick and Muir (1999, Current Op. in Micro. 2:40-45), the entire contents of which are incorporated herein by reference, describe how an autoinducer for one bacterial species may act as an inhibitor for another. These peptides can be used as agents of inhibition in the present invention. There are numerous other references citing inhibitors that a person skilled in the art would recognize as being useful in the present invention. Other quorum sensing autoinducer molecules have been described, such as gamma-butyrolactone from Streptomyces and 2-heptyl-3-hydroxy-4-quinolone from Pseudomonas aeruginosa. It is likely that additional quorum sensing systems have not yet been described.

Co-crystal structures solved by x-ray crystallography and nuclear magnetic resonance (NMR) of TraR from A. tumefaciens and another LuxR family member, SdiA from Escherichia coli, have shown these two proteins share a common overall two-domain structure and employ a conserved mechanism for binding their cognate AHL ligands (Yao et al., 2006, Mol. Biol., 13:262-73; Vannini et al, 2002, Acta Crystallogr. D. Biol. Crystallogr., 58:1362-1364; Zhang et al., 2002, Nature, 417:971-974). Based on these results and observed protein sequence similarity among many additional members of the LuxR family, it has been predicted that all LuxR proteins share a related mechanism for binding their cognate AHL ligands, as exemplified by the solved co-crystal structures of TraR and SdiAJ. (Yao et al., 2006, Mol. Biol., 13:262-73).

LuxR-type proteins contain two structural domains. The N-terminal domain (˜160 amino acid residues) mediates AHL binding, and also participates in protein dimerization. The C-terminal domain (˜60 amino acid residues) contains a helix-turn-helix DNA binding motif that mediates binding and recognition of specific DNA sequences. The TraR co-crystal structure demonstrates the direct interaction between the C-terminal domain of TraR and its cognate DNA binding site (Vannini et al, 2002, Acta Crystallogr. D. Biol. Crystallogr., 58:1362-1364; Zhang et al., 2002, Nature, 417:971-974).

LuxR-type proteins undergo a conformational change upon binding to an AHL ligand which is thought to function as a protein folding “switch”. The protein can only bind appropriate DNA sequences and stimulate transcription of its specific regulated genes when the structural switch is in the proper conformational state (Yao et al., 2006, Mol. Biol., 13:262-273).

It is believed that some bacteria, including the Gram negative bacterium Zymomonas mobilis, employ a LuxR-type protein to sense the cell density of their population through interactions with an AHL signaling molecule. Identifying the LuxR homologue produced by Z. mobilis and modifying the protein to disrupt the response of the microorganism to AHL molecules, would allow the population of these bacteria to grow to higher cell densities than they otherwise would.

Therefore, in one aspect of the invention, through heterologous expression of an AHL-degrading enzyme, a derivative bacterial strain can be created that does not respond, or gives an altered response (e.g. to a lesser degree, or with different timing), to the presence of AHL signal molecules. By way of a non-limiting example, if a bacterial strain naturally limits its growth in response to AHL signal molecules, heterologous expression of an AHL-degrading enzyme can allow the bacterial strain to grow to higher cell density.

In another aspect of the invention, if expression of an AHL-degrading enzyme is under the control of a conditional promoter (e.g. the lactose operon promoter and cognate Lad repressor protein) that can be modulated by addition of an appropriate inducer molecule (e.g. lactose or IPTG) to the media, and expression of the AHL-degrading enzyme can be controlled by appropriate choice of inducer concentration added to the growth media. This embodiment provides a system that can be fine-tuned, and one in which the response of the host strain to AHL signal molecules is modulated by differential expression of the AHL-degrading enzyme. For example, the addition of different concentrations of inducer can allow the bacteria to grow to different final cell densities, as may be desired according to the invention.

Disruption of a quorum sensing system can be accomplished by contacting a population of microorganisms with an exogenous agent that results in the modification or alteration of at least one endogenous pathway affecting quorum sensing control in the microorganism, by genetically modifying a microorganism, or by using combinations of at least one exogenous agent and at least one genetic modification. Disruption of quorum sensing systems can be accomplished, for example, at the levels of autoinducer stability, autoinducer efficacy, autoinducer production, autoinducer receptor stability, autoinducer receptor efficacy, autoinducer receptor production, autoinducer receptor binding, autoinducer receptor signaling, and responsiveness to autoinducer signaling. It is likely that additional quorum sensing systems have not yet been described. Using the methods and compositions described herein, it would be possible for one skilled in the art to quench known, and heretofore unknown, quorum sensing systems to affect the regulation of cell density.

Any combination of agents and genetic modifications can be used to disrupt a quorum sensing system. By way of a non-limiting example, an agent for a Type 1 autoinducer, an agent for a Type 2 autoinducer and an agent for a peptide autoinducer may each be used alone or in various combinations and applied to either an unmodified organism or a genetically modified organism.

In another embodiment, the invention comprises a genetically modified microorganism comprising at least one mutation to the gene encoding a LuxR-type protein, wherein the mutation modulates at least one of

a. the binding of a LuxR-type protein to DNA;

b. the binding of a LuxR-type protein to AHL; and

c. the protein folding switch of a LuxR-type protein;

wherein the mutation confers upon the genetically modified microorganism the ability to grow to a greater cell density than in the absence of the mutation. In another aspect, the mutation confers upon the genetically modified microorganism the ability to achieve a higher volumetric productivity for a fermentation product produced by the microorganism than exists in the absence of the mutation.

Other LuxR-type proteins include, but are not limited to, TraR from A. tumefaciens and SdiA from E. coli. As described in detail elsewhere herein, the present invention provides methods of identifying other proteins useful in the compositions and methods of the invention.

Volumetric productivity, as defined herein, is a measure of a product obtained in a unit volume within a unit of time. Therefore, the volumetric productivity of a microorganism cell culture, wherein the microorganism comprises at least one mutation to the gene encoding a LuxR-type protein, can be determined by ascertaining the amount of a fermentation product produced per unit volume of culture per unit time. An increased amount of volumetric productivity—i.e., an increased production of fermentation product per unit volume per unit time—in a genetically modified microorganism is an indication that the genetic modification is one which increases the volumetric productivity of the organism according to the present invention.

A greater cell density can be ascertained by any one of many ways well-known in the art, or in way or with a method yet to be discovered. All such methods of measuring and/or characterizing the density of a microorganism population are encompassed by the present invention. By way of several non-limiting examples, the density of a cell population can be ascertained by measuring the optical density of the culture, by cell counting, or by measurement of a reference parameter, such as conductivity or pH of the culture.

Increased cell density of a microorganism culture according to the present invention is also a comparison of the cell density of a culture of a particular microorganism with respect to a selected unit volume. For example, a microorganism cell culture is said to be at a greater density, prepared according to the present invention, is said to have a greater cell density when the cell culture contains more cells per unit volume. By way of a non-limiting example, the optical density of a one-liter culture is 1.5, wherein the optical density of a one liter culture of the same microorganism when not prepared according to the present invention is only 1.0, is representative of a cell culture having a greater density. The skilled artisan will understand that this comparison, and the present invention as a whole, applies to many various microorganisms, culture conditions, cell densities, and methods of measuring cell density.

Generally, the present invention features a method of modulating quorum sensing by engineering a microorganism to conduct this modulation. In one aspect, the modulation is quorum sensing quenching, as described herein. According to the invention, a microorganism can be engineered by one or more of the following: altering the genetic material of the microorganism, adding exogenous genetic material to the microorganism, altering the culture conditions of the microorganism, altering the nutrients available to the microorganism, altering the environmental signals available to the microorganism (e.g., temperature, pH, ionic strength, pressure, light, and the like), among other things.

In an embodiment, a genetically engineered microorganism is engineered to express one or more proteins, wherein the expressed proteins are responsible for modulating the quorum sensing pathway of the microorganism. In one aspect, an expressed protein is an enzyme. In an embodiment, an expressed protein can act on a component of one or more quorum sensing pathways in order to quench such pathways. In another embodiment, a protein can directly play a role in one or more quorum sensing pathways. By way of a non-limiting example, an expressed protein can bind with one or more components of a quorum sensing pathway, effectively removing the component of the quorum sensing pathway, thereby quenching the quorum sensing pathway.

The presence of an expressed protein can serve to quench the pathway by altering the natural flux through the pathway, or by redirecting the pathway, among other things. In yet another embodiment, an expressed enzyme can catalyze the production of a compound which is a quorum sensing quencher. In still another embodiment, an expressed enzyme can catalyze the modification, disruption, or elimination of a compound which is a quorum sensing molecule, or a molecule which is required for signaling through a quorum sensing pathway. Such an enzyme is therefore a quorum sensing quencher according to the present invention, and acts by disrupting or removing a quorum sensing molecule from a quorum sensing pathway.

In an embodiment of the invention, the quenching agent is an enzyme that catalyzes a reaction with the acyl homoserine lactone autoinducer. Examples of classes of enzymes include esterases, lipases, lactonases, proteases, peptidases, aminoacylases or carboxypeptidases. As will be understood by the skilled artisan, many enzymes comprising these classes are commercially available.

In an aspect, the invention relates to a method for interfering with, disrupting, removing, inhibiting or dis-enabling the acyl homoserine lactone (AHSL) chemical signals (autoinducers) which facilitate Type 1 quorum sensing in many Gram negative bacteria.

In another embodiment of the invention, by way of a non-limiting example, AHSL signals may be disrupted using a quorum sensing quenching agent produced by a microorganism that is engineered to produce the agent. That is, the microorganism may be genetically modified so that the microorganism produces an agent, wherein the agent a) opens the lactone ring, b) hydrolyzes the peptide bond, or c) modifies the acyl chain of an AHSL autoinducer.

For example, it has been demonstrated that enzymes can degrade AHSLs. Lactonase has been shown to inactivate oxohexanoyl-, oxodecanoyl- and oxooctanoyl-homoserine lactones (Dong et al., PNAS USA 97:3526-331, 2000 and Nature 411:813-817, 2001). Similarly, it has been demonstrated that a strain of Variovorax paradoxus can utilize several acyl homoserine lactones for growth; it is believed that the ring is enzymatically cleaved allowing the acyl chain and lactone ring to be used as sources of energy and nitrogen, respectively (Leadbetter and Greenberg, J. Bacteriology, 182:6921-6926). In another embodiment, the agent is a chemical other than an enzyme that catalyzes a reaction with the autoinducer molecule, such that the structure of the autoinducer is modified and the autoinducer becomes non-functional. Addition of sodium hydroxide or other base to raise the pH to greater than 8 is known to hydrolyze the lactone ring, thereby degrading the AHSL.

In an embodiment of the invention, the agent is a chemical that inhibits biosynthesis of the acylhomoserine lactone autoinducer, such as by inhibiting the luxI protein, an analog thereof, or a protein exhibiting a similar function. Examples of such an agent include cycloleucine or (2S,4S)-2-amino-4,5-epoxy pentanoic acid, inhibitors of S-adenosylmethionine synthesis. In another embodiment of the invention, the agent is a chemical that inhibits binding of the acyl homoserine lactone autoinducer to its receptor, thus blocking transcription of quorum sensing regulated genes. An example of such a chemical is an antibody that specifically binds to the receptor; the antibody may be polyclonal or monoclonal and can be prepared using methods that are well known in the art. An additional example of such a chemical is an analog of the AHSL itself. Halogenated furanones from the red alga Delisea pulchra which inhibit binding of the AHSL to the receptor that regulates swarming in Serratia liquefaciens are an example of an analog of an AHSL (Rasmussen et al., Microbiology, 146:3237-3244, 2000).

In another embodiment, an antibody can be used to bind a quorum sensing signaling molecule. In one aspect, an antibody is used to bind a quorum sensing signaling peptide in order to quench quorum sensing. In another aspect, an antibody is used to bind a quorum sensing signaling small molecule in order to quench quorum sensing. By way of a non-limiting example, an antibody specific for an AHSL can be used to bind the AHSL and quench quorum sensing in an organism.

It will be understood that, according to the present invention, such agents may be produced by engineering a microorganism to produce such agents. In one embodiment, the microorganism can be engineered to produce an enzyme which catalyzes the production of such agent, either through synthesis or through breakdown of another molecule. In accordance with the invention, such product may be produced directly, or indirectly, through a pathway leading to production of the agent. By way of a non-limiting example, a microorganism can be engineered to produce an enzyme, wherein the enzyme acts upon a compound taken up by the microorganism in culture, in order to produce the final useful quorum sensing quenching agent. An inactive “precursor” compound can be added to the microorganism culture and internalized by the microorganism, at which point the enzyme which is engineered into the microorganism converts the precursor compound into an active quorum sensing quenching agent.

In another embodiment, the invention includes a method for interfering with, disrupting, removing, inhibiting or disabling Type 2 quorum sensing. In one embodiment, the quenching agent is an enzyme that catalyzes a reaction with the Type 2 quorum sensing autoinducer, 4-hydroxy-5-methyl-2H-furan-3-one, 4,5-dihydroxy-2-cyclopent-en-1-one or an analog. In another embodiment, the agent is a chemical that disrupts the Type 2 autoinducer.

In yet another embodiment, the quenching agent is a chemical that inhibits biosynthesis of the Type 2 quorum sensing autoinducer. Agents inhibiting the biosynthesis of the Type 2 autoinducer can modify the biosynthetic enzymes themselves. Alternatively the agent can be an analog of one of the biosynthetic precursors of one of the enzymes. For example, the agent can be an analog of methionine, S-adenosyl homocysteine, or S-ribosylhomocysteine, thus preventing binding of these molecules to the appropriate enzyme and biosynthesis of the autoinducer. As set forth in detail elsewhere herein, such agents may be produced by engineering a microorganism to produce such agents.

In another embodiment of the invention, the quenching agent is a chemical that inhibits binding of the Type 2 quorum sensing autoinducer to its receptor. The agent can be a chemical that modifies luxP or luxQ, or proteins that carry out similar functions in other organisms. Similarly an agent can inhibit Type 2 quorum sensing by modifying luxO, luxR or the repressor protein, or any of the proteins that carry out similar functions in other organisms. The agent can also bind to the autoinducer receptor or other proteins involved in signal transduction between the autoinducer and the quorum sensing-controlled genes; an example is an antibody that binds to one of the proteins involved. In another embodiment, the agent can be an analog of the Type 2 autoinducer molecule, such as a modified furanone.

In one embodiment, a quorum sensing quenching agent precursor of the present invention is preferably soluble in water and may be applied or delivered with an acceptable carrier system. A composition comprising an precursor of the invention may be applied or delivered with a suitable carrier system such that the agent may be dispersed or dissolved in a stable manner so that the agent, when it is administered directly or indirectly, is present in a form in which it is available in a particularly advantageous way, as set forth in detail elsewhere herein. That is, the manner or state in which the precursor is delivered is sufficient to induce or modify the microorganism such that the microorganism can circumvent and/or inhibit one or more quorum sensing pathways, as described in detail elsewhere herein.

In another aspect, separate precursors of the present invention may be pre-blended or each component may be added separately to the same environment according to a predetermined dosage for the purpose of achieving the desired concentration level of the treatment components, with the proviso that the components eventually come into intimate admixture with one another.

In another embodiment, the invention relates to a method of interfering with, disrupting, removing, inhibiting or dis-enabling peptide-regulated quorum sensing by Gram positive bacteria. Many Gram positive bacteria use secreted peptides as autoinducers. In one embodiment, quorum sensing by Gram positive bacteria is inhibited by an enzyme that catalyzes a reaction with the peptide autoinducer. Examples of such enzymes include but are not limited to proteases, peptidases and deaminases. In some Gram positive organisms, such as Staphylococcus, the peptide contains a thiolactone ring; these autoinducers may also be disrupted by an enzyme catalyzing a reaction with the thiol bond, such as a thiol reductase. In another embodiment of the invention, the quenching agent is a chemical that disrupts the structure of the autoinducer peptide such as by modifying carboxyl or amide groups. In still another embodiment of the invention, the agent is an antibody that binds to the autoinducer peptide, thus preventing binding of the peptide to its receptor protein. The antibody may also bind an autoinducer propeptide, thus preventing post-translational processing to the active autoinducer. In an aspect of the invention, peptidomimetics, such as β-peptides, may also inhibit binding of a peptide to its receptor.

In another embodiment of the invention, the agent is a chemical that inhibits the biosynthesis of the autoinducer peptide. The agent may, for example, inhibit transcription of the peptide or its propeptide (in the case of autoinducers that are post-translationally modified). The agent may inhibit the cleavage of the autoinducer peptide from its pro-peptide.

In another embodiment, the agent is a chemical that inhibits the binding of the peptide to its receptor protein. The agent may be a chemical or enzyme that modifies the receptor or binds to the receptor, thereby inactivating it; an example is an antibody specific for the receptor which disrupts binding of the autoinducer to the receptor. In another embodiment, the agent is an analog of the autoinducer peptide which binds to the receptor, thereby preventing binding of the autoinducer. Novick and Muir (1999, Current Op. in Micro. 2:40-45), the entire contents of which is herein incorporated by reference, describes how an autoinducer for one bacterial species may act as an inhibitor for another. These peptides can be used as agents of inhibition in the present invention. There are numerous other references citing inhibitors that a person skilled in the art would recognize as being useful in the present invention (See, e.g., Lin et al., 2003 Mol. Microbiol. 47:849-860).

Other quorum sensing autoinducer molecules have been described, such as gamma-butyrolactone from Streptomyces and 2-heptyl-3-hydroxy-4-quinolone from Pseudomonas aeruginosa. However, it is likely that additional quorum sensing systems have not yet been described. Using the methods described above, it would be possible for one skilled in the art to identify, characterize, and/or disrupt these quorum sensing systems in order to allow colony formation or culture growth by organisms that regulate cell density by using quorum sensing. Therefore, the present invention also encompasses methods and compositions comprising quorum sensing systems yet to be discovered.

Any combination of agents as described herein can be used to interfere with, disrupt, remove, or disable or inhibit quorum sensing. By way of a nonlimiting example, an agent for the Type 1 autoinducer, an agent for the Type 2 autoinducer and an agent for the peptide autoinducer can be combined and used to regulate quorum sensing in a single reaction mixture according to the invention.

A protein quorum sensing quenching agent, including enzymes, according to the invention is preferably a known protein. The proteins described herein, useful in the compositions and methods according to the present invention, can be produced in various ways, as will be understood by the skilled artisan. In an embodiment, a protein can be added to a cell exogenously (e.g., added to the cell culture and taken up by the cells in culture). By way of a non-limiting example, a microorganism can be engineered to express an enzyme which acts upon a protein, wherein the protein is a precursor to a quorum sensing quenching agent. The precursor protein can be added to the culture medium of an engineered microorganism, internalized by the microorganism, and then acted upon by the enzyme produced by the engineered microorganism, to produce an active quorum sensing quenching agent.

By way of another non-limiting example, a known microorganism can be engineered to express a first protein which dimerizes with a precursor protein, wherein the expressed first protein—precursor protein dimer forms an active quorum sensing quenching agent. The precursor protein can be added to the culture medium of the microorganism, and internalized, whereupon it dimerizes with the expressed first protein. Alternatively, the precursor protein may be co-expressed within the microorganism with the first protein, and upon co-expression, the first protein and the precursor protein dimerize to form the active quorum sensing quenching agent.

In another embodiment of the invention, a nucleic acid plasmid can be added to a cell by any means known in the art, wherein the plasmid encodes a desired protein. The protein is subsequently expressed from the plasmid. In another embodiment, a nucleic acid encoding a protein of interest may be integrated into the chromosome of the target microorganism, and the encoded protein subsequently expressed there from.

Additionally, expressed proteins may be used according to the present invention in any number of ways, including, but not limited to, directly binding with a member of a quorum sensing pathway, enzymatically acting upon a member of a quorum sensing pathway, interacting with or acting upon a molecule that regulates expression of one or more members of a quorum sensing pathway, and by forming a multimeric complex with a third molecule, wherein the multimeric complex is responsible for the quorum sensing quenching. It will be understood that any combination of the above methods may also be used according to the invention.

Methods of increasing volumetric productivity according to the present invention are useful for many purposes, as set forth in detail elsewhere herein. In an aspect of the invention, the increased volumetric productivity is useful for obtaining an increased or greater amount of fermentation product derived from the microorganism. This is because a greater volumetric productivity of microorganism producing a fermentation product provides a higher or greater yield of that fermentation product per unit volume of cell culture.

Methods of increasing cell density according to the present invention are also useful for many purposes, as set forth in detail elsewhere herein. In an aspect of the invention, the increased cell density is useful for obtaining an increased or greater amount of fermentation product derived from the microorganism. This is because a higher density of microorganism producing a fermentation product will result in a higher or greater yield of that fermentation product per unit volume of cell culture. In this way, methods of increasing cell density according to the invention provide greater volumetric productivity

In one aspect of the invention, a microorganism is one which naturally produces a desired fermentation product. In another aspect, a microorganism is one which is genetically modified to produce a desired fermentation product. In an embodiment, a microorganism produces more than one fermentation product, wherein each fermentation product may either be produced in the microorganism naturally, or by genetic modification of the microorganism. Such genetic modifications are discussed in detail elsewhere herein.

A great many texts are available which describe procedures for expressing foreign genes. Also, catalogs list cloning vectors which can be used for various organisms including Gram-positive bacteria. Catalogs from which these cloning vectors can be ordered are readily available and well known to those skilled in the art. See, for example, Marino (1989) BioPharm. 2:18-33; Vectors: A Survey of Molecular Cloning Vectors and Their Uses (Butterworths 1988).

In another embodiment of the invention, chromosomal integration of foreign genes can offer several advantages over plasmid-based constructions, the latter having certain limitations for commercial processes. Initial selections of recombinants can be made on 20 mg chloramphenicol (“Cm”)/liter plates to allow growth after single copy integration. These constructs may be obtained at a very low frequency. Higher level expression may be achieved as a single step by selection on plates containing 600 to 1000 mg Cm/liter. Such strains have proven very stable. Testing of certain wild strains indicates that electroporation improves plasmid delivery and may reduce the effort required to achieve integrations.

Those skilled in this art will appreciate that many microorganisms are suitable for use in the present invention. In one aspect, a microorganism useful in the invention is a bacterium. In another aspect, a microorganism useful in the invention is a yeast.

Those skilled in the art will appreciate that a number of modifications can be made to the methods and materials exemplified herein. For example, a variety of promoters can be utilized to drive expression of the heterologous genes in the Gram-positive recombinant host. The skilled artisan, having the benefit of the instant disclosure, will be able to readily choose and utilize any one of the various promoters available for this purpose. Similarly, skilled artisans, as a matter of routine preference, may utilize a higher copy number plasmid or, as described herein, chromosomal integration of the desired genes. Further optimization can be readily achieved by replacing the ribosomal binding site on genes of interest with a native ribosomal binding site from the Gram-positive host. Specifically, in the case of a Bacillus host, the operon can be modified to include the binding site from a Bacillus gene. Finally, it is a matter of routine laboratory practice to mutate with chemicals or radiation to create and select mutants with higher levels of expression. Aldehyde indicator plates or pyruvate decarboxylase activity stains can be conveniently used to identify strains with useful mutations.

### II. Fermentation

The fermentation of microorganisms for the production of natural products is a widely known application of biocatalysis. Industrial microorganisms effect the multistep conversion of renewable feedstocks to high value chemical products in a single reactor and in so doing catalyze a multi-billion dollar industry. Fermentation products range from fine and commodity chemicals such as ethanol, lactic acid, amino acids and vitamins, to high value small molecule pharmaceuticals, protein pharmaceuticals, and industrial enzymes.

Success in bringing these products to market and success in competing in the market depends partly on continuous improvement of the whole cell biocatalysts.

Improvements include the ability to grow microorganisms to a greater cell density, increased yield of desired products, increased amount of volumetric productivity, removal of unwanted co-metabolites, improved utilizaton of inexpensive carbon and nitrogen sources, and adaptation to fermenter conditions, increased production of a primary metabolite, increased production of a secondary metabolite, increased tolerance to acidic conditions, increased tolerance to basic conditions, increased tolerance to organic solvents, increased tolerance to high salt conditions and increased tolerance to high or low temperatures. Shortcomings in any of these areas can result in high manufacturing costs, inability to capture or maintain market share, and failure of bringing promising products to market.

The methods and compositions of the present invention can be adapted to conventional fermentation bioreactors (e.g., batch, fed-batch, cell recycle, and continuous fermentation) to improve the fermentation process. The use of a type of mutagenesis methodology to disrupt a quorum sensing gene provides a strategy to increase cell density.

The use of a cell modified to have at least one gene associated with quorum sensing disrupted in the context of in a large scale setting can improve efforts of the fermentation industry by providing a means to establish increased-density cultures where traditional fermentation methods have experienced difficulties in establishing increased-density cultures. In applications where production yield can be increased through an increase in population cell density or volumetric productivity, disruption of the quorum sensing systems of microbial populations can lead to an increase in yield. As such, the methods disclosed herein in turn increases the profitability of current fermentation processes and can facilitate the development of new products.

For obtaining a desired product from a microorganism (e.g., bacteria), the bacteria are generally cultivated in liquid media (submerged cultures) leading to excretion of the products into the liquid, from which they can be isolated. Formation of product can take place during the initial fast growth of the organism and/or during a second period in which the culture is maintained in a slow-growing or non-growing state. During such a process, the amount of product which is formed per unit of time (the productivity) is generally a function of a number of factors: the intrinsic metabolic activity of the microorganism; the physiological conditions prevailing in the culture (e.g. pH, temperature, medium composition); and the amount of microorganisms which are present in the equipment used for the process. Generally, during optimization of a fermentation process, the focus is on obtaining the highest possible productivity. One solution to this problem is obtaining a concentration of bacteria that is as high as possible. The use of a modified cell where a quorum sensing gene has been disrupted allows for the increased-density culture. This would mean that the fermentation process which includes the use of a modified cell can be operated at a higher production rate and/or achieve a higher concentration of the desired product.

The modified fermentation process can also be applied to scenarios where a large scale isolation of a recombinant polypeptide is desired. Initially, prior to expression of the polypeptide of interest in the fermentation process, the host cell containing the exogenous gene corresponding to the desired recombinant polypeptide is inoculated into the ferment or are grown under favorable growth conditions, e.g., with all of the available oxygen and carbon/energy sources (or, preferably, source), along with essential nutrients and pH control, necessary for logarithmic growth. In accordance with the invention, these conditions are maintained, e.g., by feeding concentrated glucose at a rate that controls dissolved oxygen content at a set point, until the host cells expand in culture to the desired number or cell density.

After reaching target cell density, further manipulations of the fermentation can occur. The first is to provide the signal to the host cells in order to induce expression of the polypeptide by the host cells. The second manipulation (which can result from the first) is to downshift or reduce the host cell metabolic rate. Since during logarithmic growth the metabolic rate is directly proportional to availability of oxygen and a carbon/energy source, reducing the levels of available oxygen or carbon/energy sources, or both, can reduce metabolic rate. Manipulation of ferment or operating parameters, such as agitation rate or back pressure, as well as reducing O₂ pressure, can modulate available oxygen levels. Reducing concentration or delivery rate, or both, of the carbon/energy source(s) has a similar effect. Furthermore, depending on the nature of the expression system, induction of expression can lead to dramatic decrease in metabolic rate.

The polypeptide of interest preferably is recovered from the periplasm or culture medium as a secreted polypeptide, although it also may be recovered from host cell lysates when directly expressed without a secretory signal. Alternatively, the cells or portions thereof may be used as biocatalysts or for other functions without substantial purification.

It is often preferred to purify the polypeptide of interest from recombinant cell proteins or polypeptides to obtain preparations that are substantially homogeneous as to the polypeptide of interest. As a first step, the culture medium or lysate is centrifuged to remove particulate cell debris. The membrane and soluble protein fractions may then be separated if necessary. The polypeptide may then be purified from the soluble protein fraction and from the membrane fraction of the culture lysate, depending on whether the polypeptide is membrane bound, is soluble, or is present in an aggregated form. The polypeptide thereafter is solubilized and folded, if necessary, and is purified from contaminant soluble proteins and polypeptides, with the following procedures being exemplary of suitable purification procedures: fractionation on immunoaffinity or ion-exchange columns; ethanol precipitation; reverse phase HPLC; chromatography on silica or on a cation-exchange resin such as DEAE; chromatofocusing; SDS-PAGE; ammonium sulfate precipitation; and gel filtration using, for example, Sephadex G-75.

### III. Random Disruption

The invention provides methods of modifying a cell, such as Z. moblilis in the context of screening a library (e.g. gene disruption library) to acquire or evolve a cell to have desired function. A desirable function is the ability for the mutant to grow at a higher density compared to another wise Z. moblilis not genetically modified. Such a function can be observed when quorum sensing system is disrupted in the cell. Accordingly, the invention encompasses identifying a gene from the mutant, wherein the gene is involved in limiting cell density.

Genes important for growth control (e.g., quorum sensing) in a microorganism such as Z. mobilis can be discovered and identified by using type of gene disruption library (e.g., a transposon-based mutagenesis strategy). The library encompasses derivative strains containing random “knockout” mutations. Mutant stains are screened for growth effects by measuring the optical density of growing cultures. Strains exhibiting at least (i) increased cell density in stationary phase, (ii) increased growth rate, or (iii) a reduced lag phase duration are selected for DNA sequence analysis.

Accordingly, the invention also encompasses identification of genes that when disrupted allows for the bacteria to be more cultivable. For example, the invention allows for identification of genes that are essential for limiting cell density and disruption of such a gene in a cell allows the cell to grow at an increase-density.

Such methods of randomly disrupting a gene in a cell includes, e.g., introducing a library of DNA fragments into a plurality of cells, whereby at least one of the fragments undergoes recombination with a segment in the genome or an episome of the cells to produce modified cells. The modified cells are then screened for modified or recombined cells that have evolved toward acquisition of the desired function. DNA from the modified cells that have evolved toward the desired function is then optionally recombined with a further library of DNA fragments, at least one of which undergoes recombination with a segment in the genome or the episome of the modified cells to produce further modified cells. The further modified cells are then screened for further modified cells that have further evolved toward acquisition of the desired function. Having the cells to go through a second round of modification or otherwise subjecting the cells to more than one round of modification allows for the identification of genes that cooperate or have a synergistic effect with one another on modulating quorum sensing. Steps of recombination and screening/selection are repeated as required until the further modified cells have acquired the desired function. As such, the invention also includes identification of a combination of genes that collectively regulate quorum sensing. Disruption of a combination of genes that regulate quorum sensing and in some instance, enhance the disruption of quorum sensing is also contemplated in the invention.

The present invention generally relates to the identification and discovery of quorum sensing genes accomplished by screening a gene disruption library and determining whether the disrupted gene modulates cell grow. Preferably, a cell containing the disrupted gene allows the cell to be more cultivatable. However, the invention can also encompasses screening a library for the identification of regulatory components of a gene involved in cell grow.

In addition to the aspect of screening a gene disruption library to identify quorum sensing genes, the invention includes development of partial sequence data on quorum sensing genes and disrupting at least one quorum sensing gene in order to disable quorum sensing in the microorganism. One method of disrupting a gene is the generic method of mutagenesis, which is a technique that aims to artificially modify the nucleotide sequence of a DNA fragment, with the intention of modifying the biological activity resulting therefrom.

### Mutagenesis

The term mutagenesis or otherwise mutation can be associated with at least three distinct modifications of a DNA fragment (i.e., deletion, insertion, and substitution). Deletion corresponds to removal of one or more nucleotides from the DNA fragment of interest; insertion corresponds to addition of same; substitution corresponds to replacement of one or more bases with a same number of bases of different nature.

In this context of seeking mutants having acquired a novel property or having an improved existing property, mutagenesis constitutes a first step and creates diversity. In a second step, diversity is then screened by means of a functional test, so as to isolate a mutant containing a mutation in a gene that confers an improved or desired property.

However, the number of mutants having to be screened can be reduced if the library was generated or chosen based on some rationally basis. For example, a gene disruption library can be generated based on known quorum sensing genes and related genes. In this case it is expected that the frequency of desired mutants in these semirational libraries are higher than if diversity were generated solely on a random basis.

Various mutagenesis methods have been developed and can be adapted to identifying a quorum sensing gene and/or disrupting quorum sensing in a microorganism. Mutagenesis methods can be divided into at least five main groups: random mutagenesis; mutagenesis by DNA shuffling (recombination); directed mutagenesis; saturation mutagenesis; and the like.

Mutagenesis can also include mutations that enhance the activity of a protein. For example, a library can be screened for activation mutations or otherwise mutations that enhance the ability of an inhibitor to inhibit proteins involved in quorum sensing. In essence, activation or enhanced activity of an inhibitor of quorum sensing can inhibit quorum sensing in the microorganism and as a result confers increased ability to grow to higher densities and/or achieve a higher volumetric productivity.

### Screening

The present invention includes a method of identifying a gene involved in cell growth of a microorganism. The method comprises providing a known microorganism with an exogenous nucleic acid comprising a mutant form of a gene so that the mutant nucleic acid allows the mutant microorganism to grow at higher densities and/or achieve a higher volumetric productivity. The mutation can be disruption of a gene involved in cell growth. Alternatively, the mutation can be activation of a gene involved in the regulation of genes involved in cell growth. Moreover, the mutation can be in the regulatory sequences of a genes involved in cell growth. That is, any genetic mutation that results in increase cell growth and/or achieve a higher volumetric productivity of the microorganism is encompassed in the invention. It is also contemplated that the invention allows for individually storage of each mutant and the isolation of the exogenous nucleic acid therefrom.

Preferably, the individual mutant has at least one component of the quorum sensing system disrupted therefore allowing for the mutant to grow to a greater cell density as compared to the density if the quorum sensing system was not disrupted. Accordingly, the invention also includes identification of the exogenous mutant gene that is expressed by the individual mutant or otherwise a gene of a microorganism which regulates the population density of the microorganism and/or achieve a higher volumetric productivity. This is because when armed with the sequence of the mutant gene, a skilled artisan would know how to identify the corresponding wild-type gene.

The following discussion relates to using a type of gene disruption construct and/or library that involves a transposable nucleic acid element to generate mutations in the genome of the host cell. With respect to libraries according to the invention, a library of polynucleotides or a library of transposon insertion sites is a collection of sequence information, which information is provided in either biochemical form (e.g., as a collection of polynucleotide molecules), or in electronic form (e.g., as a collection of polynucleotide sequences stored in a computer-readable form, as in a computer system and/or as part of a computer program). The sequence information of the polynucleotides can be used in a variety of ways, for instance as a resource for gene discovery, i.e., for identifying and verifying essential and important genes in a particular microorganism, or for identifying essential or important homologues in other genera or species. A polynucleotide sequence in a library can be a polynucleotide that represents an mRNA, polypeptide, or other gene product encoded by the polynucleotide, and accordingly such a polynucleotide library could be used to formulate corresponding RNA or amino acid libraries according to the sequences of the library members.

It will be appreciated that although transposons are convenient for insertionally inactivating a gene, any other known method, or method developed in the future may be used to screen for a gene associated with quorum sensing. In any event, the mutants can be screened for quorum sensing genes, as well as other classes of genes. Thus, the present invention encompasses nucleic acid elements, bacterial mutants, production methods, screening methods, and therapeutic methods.

A High-Throughput Transposon Insertion Mapping (HTTIM) strategy can be used to identify genes associated with quorum sensing. Such a strategy utilizes a transposon, which is a small, mobile DNA element that randomly inserts into the chromosome. Any transposon may be employed so long as its insertion into the chromosome is random, i.e., devoid of hot spots.

When the transposon insertion disrupts one of the essential genes in the genome, the function of that gene is lost. If the disrupted gene is associated with quorum sensing, the transposon insertion mutant is able to grow at a higher density than if the identical gene was not disrupted as a result of disruption of quorum sensing. Disruption of a gene associated with quorum sensing provides a way to cultivate microorganisms that otherwise would not have been able to be cultivated. By examining the insertion sites of a large number of transposon mutants, potentially all, of the quorum sensing genes can be identified, and previously uncultivable microorganisms can be cultivated.

In some cases, a transposon will be inserted into the genome so as to negatively or to positively affect quorum sensing. The term “negatively affects quorum sensing” means that the microorganism host with a transposon insertion has a disruption in quorum sensing and therefore is able to grow to a higher density compared to an otherwise identical microorganism host lacking the insertion. Similarly, the term “positively affect virulence” means that the bacterial host with a transposon insertion is more sensitive to quorum sensing compared to an otherwise identical microorganism host lacking the insertion.

In some embodiments, the invention covers a transposable nucleic acid element, which can be incorporated into the genome of a heterologous organism, such as bacteria Particularly contemplated is an element comprising a pair of inverted repeat sequences recognized transposase. The transposon or otherwise transposon-like element may contain other nucleic acid sequences. In further embodiments, the element has, but is not limited to, at least one screenable marker gene. The element may have, have at least, or have at most 1, 2, 3, 4, 5, 6 or more screenable marker genes, as well as promoters or other control elements for expression of polypeptides that may be encoded by the sequences. Such screenable markers may encode for polypeptides that are calorimetric, fluorescent, or enzymatic. Furthermore, an element may contain one or more selectable marker genes and/or one or more non-selectable (but screenable) marker genes. In some cases, the element has a selectable marker gene that encodes a polypeptide conferring antibiotic resistance. The resistance can be, but is not limited to, an antibiotic selected from the group consisting of erythromycin, tetracycline, spectinomycin, kanamycin, chloramphenicol, and the like.

The screenable marker can be used to identify a organism in which the element has been incorporated intrachromosomally or episomally. In particular cases, it can be used to identify microorganisms that have the element inserted into a chromosome. Alternatively, a screenable element can be used to identify or characterize nucleic acid control sequences near or at the site of integration. In some instances a screenable marker lacks a promoter so as to identify or characterize a nucleic acid sequence at the site of integration that provides a promoter sequence. The identification of enhancers can similarly be implemented. In some embodiments, the screenable marker is a gene encoding a colorimetric polypeptide, such as a green fluorescent protein.

In certain embodiments of the invention, there is a nucleic acid encoding a transposase that recognizes and transposes the transposable element of the invention. In some examples of the invention, there is a plasmid or vector containing a transposable element of the invention and/or a nucleic acid encoding a transposase that recognizes the element. Such vectors can be in a bacterium either to propagate the vector or as the target of transposon-induced mutagenesis. Moreover, both the vector containing the transposable element and a vector containing the cognate transposase may be in the same microorganism together.

The invention further encompasses a microorganism having a transposon insertion, which means the microorganism has at least one element that was transposed into its genome. The insertion could be random or pre-determined or engineered.

Microorganisms with different mutations can be created using the transposable elements of the invention. The transposon may insert itself in any site throughout the genome and is not limited to any particular place. Each individual microorganism has a mutation in a specific location. In some embodiments, the insertion is in a gene selected from the group consisting of quorum sensing gene, metabolic gene, regulatory gene, extracellular factor gene, cellular or secreted gene, and any putative gene based on the presence of an ORF and/or conservation of sequence with other organisms (“hypothetical gene” or “conserved hypothetical gene”). Alternatively, the insertion may be in a gene encoding an autoinducer, an enzyme, a structural protein, a membrane protein, transporter, symporter, or a functional RNA molecule (such as an rRNA, tRNA, tmRNA, or small RNA), and any other gene in the microorganism genome.

Random integration of the transposon or other DNA sequence allows isolation of a plurality of independently mutated microorganisms wherein a different gene is insertionally inactivated in each mutant and each mutant contains a different marker sequence. A collection of such insertion mutants is arrayed in welled microtitre dishes so that each well contains a different mutant microorganism. DNA comprising the unique marker sequence from each individual mutant microorganism (conveniently, the total DNA from the clone is used) is stored. This is done by removing a sample of the microorganism from the microtitre dish, spotting it onto a nucleic acid hybridisation membrane (such as nitrocellulose or nylon membranes), lysing the microorganism in alkali and fixing the nucleic acid to the membrane. Thus, a replica of the contents of the welled microtitre dishes is made.

Pools of the microorganisms from the welled microtitre dish are made and DNA is extracted. This DNA is used as a target for a PCR using primers that anneal to the common “arms” flanking the “tags” and the amplified DNA is labelled, for example with P₃₂. The product of the PCR is used to probe the DNA stored from each individual mutant to provide a reference hybridization pattern for the replicas of the welled microtitre dishes. This is a check that each of the individual microorganisms does, in fact, contain a marker sequence and that the marker sequence can be amplified and labelled efficiently.

Pools of transposon mutants are made to introduce into the particular environment. 96-well microtitre dishes can be used and the pool contains 96 transposon mutants. Theoretically, the lower limit for the pool is two mutants; there is no theoretical upper limit to the size of the pool.

In another aspect, the invention provides a method of identifying a gene which allows a microorganism to grow at a high density and/or achieve a higher volumetric productivity. The method comprises isolating the insertionally-inactivated gene or part thereof from the individual mutant selected following the first round of selection from gene disruption library screen. However, as discussed elsewhere herein, any type of mutation can be screened for as long as the phenotype of the mutant is the enhanced ability to grow at higher densities and/or achieve a higher volumetric productivity. Addition rounds of screen and selection can be performed. Regardless, standard molecular biology techniques can be used to isolate and characterize the genes. Methods for isolating a gene containing a unique marker are well known in the art of molecular biology. Methods for gene probing are well known in the art of molecular biology. Molecular biological methods suitable for use in the practice of the present invention are disclosed in Sambrook et al., MOLECULAR CLONING: A LABORATORY MANUAL, volumes 1-3 (3^{rd} ed., Cold Spring Harbor Press, NY 2001), which is incorporated herein by reference.

### Vectors

The invention encompasses vectors comprising the nucleic acid sequences, open reading frames and genes of the invention, as well as host cells containing such vectors. Because the quorum sensing genes identified herein can be readily isolated and the encoded gene products expressed by routine methods, the invention also provides the polypeptides encoded by those genes, as well as genes having at least about 50%, or more preferably about 60%, or more preferably about 70%, or more preferably about 80%, or more preferably about 90%, or most preferably about 95% protein sequence identity.

The term “vector” is used to refer to a nucleic acid molecule into which a nucleic acid sequence can be inserted for introduction into a cell where it can be replicated and/or expressed. A nucleic acid sequence can be “exogenous,” which means that it is foreign to the cell into which the vector is being introduced or that the sequence is homologous to a sequence in the cell but in a position within the host cell nucleic acid in which the sequence is ordinarily not found. One of skill in the art would be well equipped to construct a vector through standard recombinant techniques, which are described in Sambrook et al., MOLECULAR CLONING: A LABORATORY MANUAL, volumes 1-3 (3^{rd} ed., Cold Spring Harbor Press, NY 2001), and Ausubel et al. (1997, Current Protocols in Molecular Biology, John Wiley & Sons, New York), both incorporated herein by reference.

The term “expression vector” refers to a vector containing a nucleic acid sequence coding for at least part of a gene product capable of being transcribed. In some cases, RNA molecules may then be translated into a protein, polypeptide, or peptide. In other cases, these sequences are not translated, for example, in the production of antisense molecules or ribozymes. Expression vectors can contain a variety of “control sequences,” which refer to nucleic acid sequences necessary for the transcription and possibly translation of an operably linked coding sequence in a particular host organism.

A vector typically contains a promoter region. A “promoter” is a control sequence that is a region of a nucleic acid sequence at which initiation and rate of transcription are controlled. It may contain genetic elements at which regulatory proteins and molecules may bind such as RNA polymerase and other transcription factors. The phrases “operatively positioned,” “operatively linked,” “under control,” and “under transcriptional control” mean that a promoter is in a correct functional location and/or orientation in relation to a nucleic acid sequence to control transcriptional initiation and/or expression of that sequence. A promoter may or may not be used in conjunction with an “enhancer,” which refers to a cis-acting regulatory sequence involved in the transcriptional activation of a nucleic acid sequence.

A promoter may be one naturally associated with a gene or sequence, as may be obtained by isolating the 5′ non-coding sequences located upstream of the coding segment. Similarly, an enhancer may be one naturally associated with a nucleic acid sequence, located either downstream or upstream of that sequence. Alternatively, certain advantages will be gained by positioning the coding nucleic acid segment under the control of a recombinant or heterologous promoter, which refers to a promoter that is not normally associated with a nucleic acid sequence in its natural environment. A recombinant or heterologous enhancer refers also to an enhancer not normally associated with a nucleic acid sequence in its natural environment.

It is advantageous to employ a promoter and/or enhancer that effectively directs the expression of the DNA segment in the cell type chosen for expression. Those of skill in the art of molecular biology generally know the use of promoters, enhancers, and cell type combinations for protein expression, for example, see Sambrook et al., MOLECULAR CLONING: A LABORATORY MANUAL, volumes 1-3 (3^{rd} ed., Cold Spring Harbor Press, NY 2001). The promoters employed may be constitutive, tissue-specific, inducible, and/or useful under the appropriate conditions to direct high level expression of the introduced DNA segment, such as is advantageous to grow microorganisms to a greater cell density, increased yield of desired products, increased amount of volumetric productivity, removal of unwanted co-metabolites, improved utilizaton of inexpensive carbon and nitrogen sources, and adaptation to fermenter conditions, increased production of a primary metabolite, increased production of a secondary metabolite, increased tolerance to acidic conditions, increased tolerance to basic conditions, increased tolerance to organic solvents, increased tolerance to high salt conditions, increased tolerance to high or low temperatures, etc.

In order to propagate a vector in a host cell, it may contain one or more origins of replication sites (often termed “ori”), which is a specific nucleic acid sequence at which replication is initiated. The origin of replication may optionally be active or non-active at specific temperatures, i.e., temperature sensitive.

In certain embodiments of the invention, the cells contain nucleic acid construct of the present invention, a cell may be identified in vitro by including a marker in the expression vector. Such markers would confer an identifiable change to the cell permitting easy identification of cells containing the expression vector. Generally, a selectable marker is one that confers a property that allows for selection. A positive selectable marker is one in which the presence of the marker allows for its selection, while a negative selectable marker is one in which its presence prevents its selection. An example of a positive selectable marker is a drug resistance marker.

Usually the inclusion of a drug selection marker aids in the cloning and identification of transformants, for example, genes that confer resistance to neomycin, puromycin, hygromycin, DHFR, GPT, zeocin and histidinol are useful selectable markers. In addition to markers conferring a phenotype that allows for the discrimination of transformants based on the implementation of conditions, other types of markers including screenable markers such as GFP, whose basis is colorimetric analysis, are also contemplated. Alternatively, screenable enzymes such as herpes simplex virus thymidine kinase (tic) or chloramphenicol acetyltransferase (CAT) may be utilized. One of skill in the art would also know how to employ fluorescent or chemilluminescent markers, possibly in conjunction with FACS analysis. The marker used is not believed to be important, so long as it is capable of being expressed simultaneously with the nucleic acid encoding a gene product. Further examples of selectable and screenable markers are well known to one of skill in the art.

In certain aspects of the present invention, cells containing transposon are identified with specific markers. Such markers confer on their recombinant hosts a readily detectable phenotype that emerges only under conditions where the transposon has integrated into the genome of the host. Generally reporter genes encode a polypeptide not otherwise produced by the host cell which is detectable by analysis of the cell culture, e.g., by drug-resistance, or fluorometric or spectrophotometric analysis of the cell culture.

With respect to a selection marker, contemplated for use in the present invention is green fluorescent protein (GFP) as a marker for transgene expression. The use of GFP does not need exogenously added substrates, only irradiation by near UV or blue light, and thus has significant potential for use in monitoring gene expression in living cells. Other particular examples are the enzymes firefly and bacterial luciferase, and the bacterial enzymes β-galactosidase and β-glucuronidase. Other marker genes within this class are well known to those of skill in the art, and are suitable for use in the present invention.

Another class of reporter genes which confer detectable characteristics on a host cell are those which encode polypeptides, generally enzymes, which render their transformants resistant against toxins. Examples of this class of reporter genes are the neo gene which protects host cells against toxic levels of the antibiotic G418, the gene conferring streptomycin resistance, the gene conferring hygromycin B resistance, a gene encoding dihydrofolate reductase, which confers resistance to methotrexate, the enzyme HPRT, along with many others well known in the art. Chloramphenicol acetyltransferase (CAT) confer resistance to chloramphenicol, and the β-lactamase gene confers ampicillin resistance.

In accordance with the present invention, nucleic acid sequences are transferred into a desired cell (e.g., bacterial cells) using standard methodologies known to those of ordinary skill in the art. In certain embodiments of the present invention, the vector or otherwise construct is introduced into the cell via electroporation. Electroporation involves the exposure of a suspension of cells and DNA to a high-voltage electric discharge. Electroporation works well with bacteria.

In other embodiments of the present invention, the construct is introduced to the cells using calcium phosphate precipitation. In another embodiment, the expression construct is delivered into the cell using DEAE-dextran followed by polyethylene glycol.

Another embodiment of the invention includes transferring a naked DNA expression construct into cells by way of particle bombardment. This method depends on the ability to accelerate DNA-coated microprojectiles to a high velocity allowing them to pierce cell membranes and enter cells without killing them (Klein et al., 1992, Biotechnology 24:384-6). Several devices for accelerating small particles have been developed. One such device relies on a high voltage discharge to generate an electrical current, which in turn provides the motive force. The microprojectiles used have consisted of biologically inert substances such as tungsten or gold beads. Further embodiments of the present invention include the introduction of the expression construct by direct microinjection or sonication loading.

Chemical means for introducing a construct into a host cell include colloidal dispersion systems, such as macromolecule complexes, nanocapsules, microspheres, beads, and lipid-based systems including oil-in-water emulsions, micelles, mixed micelles, and liposomes. The preparation and use of such systems is well known in the art.

Regardless of the method used to introduce exogenous nucleic acids into a host cell, in order to confirm the presence of the recombinant DNA sequence in the host cell, a variety of assays may be performed. Such assays include, for example, “molecular biological” assays well known to those of skill in the art, such as Southern and Northern blotting, RT-PCR and PCR; “biochemical” assays, such as detecting the presence or absence of a particular peptide, e.g., by immunological means (ELISAs and Western blots) or by assays described herein to identify agents falling within the scope of the invention.

### Selection

Following the introduction of a polynucleotide into a host cell (e.g., gene disruption library, transposon mutagenesis, activating mutation, and the like) a skilled artisan would select appropriate transformed strains using a variety of criteria. By way of a non-limiting example, the screening process is discussed in the text of a transposon mutagenesis procedure. At a first level of selection, one can simply wish to identify those host cells that have taken up the transposon. At a second level, one can look for particular functional attributes of the transformants, presumably based on the site of integration of the transposon and the effect caused thereby. And at a third level, it may be desirable to identify the precise nature and location of the integration, optionally for the purpose of identifying genes or assigning function thereof. Various methods for achieving each of these selections are described below.

In one embodiment, the transposon can contain a selectable marker. A selectable marker is an element, usually a polypeptide, that permits ready identification and selection of a transformant. A classic and often used selectable marker is a gene encoding a protein that confers resistance to an antibiotic. Antibiotics useful in selection procedures are well known to those of skill in the art and include chloramphenicol, ampicillin, hygromycin B, puromycin, zeocin, G418, and others.

Methods for antibiotic selection are well known to those of skill in the art. Appropriate concentrations of antibiotics are well known based on the desired purpose. For example, bacterial cells are cultured with antibiotics under other conditions otherwise suitable for growth of host cells. Selection may also involve using increasing concentrations of antibiotic with successive rounds of more rigorous selection. Selection may be in broth culture or plated on agar, or successive combinations of both.

In another embodiment, transformants may be selected on the basis of expression of a fluorescent or a luminescent marker. The marker may be red fluorescent protein, green fluorescent protein, cyan fluorescent protein, or variants thereof. Luminescent markers include luciferase. In this aspect, the present invention may take advantage of fluorescence-activated cell sorting (FACS). This technique utilizes a machine that can rapidly separate cells in a suspension on the basis of size and the color of their fluorescence. Generally, a cell suspension containing cells labeled with a fluorescent protein is directed into a thin stream so that all the cells pass in single file. This stream emerges from a nozzle vibrating at some 40,000 cycles per second which breaks the stream into 40,000 discrete droplets each second. Some of these may contain a cell. A laser beam is directed at the stream just before it breaks up into droplets. As each labeled cell passes through the beam, its resulting fluorescence is detected by a photocell. If the signals from the two detectors meet either of the criteria set for fluorescence and size, an electrical charge (+ or −) is given to the stream. The droplets retain this charge as they pass between a pair of charged metal plates. Positively-charged drops are attracted to the negatively-charged plate and vice versa. Uncharged droplets (those that contain no cell or a cell that fails to meet the desired criteria of fluorescence and size) pass straight into a third container and are later discarded. This apparatus can sort as many as 300,000 cells per minute.

In another aspect, the present invention encompasses methods for identifying functional differences between mutants and their parental strains. The functional differences maybe any of a variety of different traits including growth, doubling time, nutrient dependence, drug susceptibility or pathogenicity.

In another embodiment, the present invention encompasses the use of transposons that contain segments encoding marker genes, but lacking the regulatory sequences needed to effect transcription. However, if the transposon integrates near a promoter sequence, the adjacent promoter may drive expression of the otherwise promoter-less marker gene, thereby permitting identification/selection as described elsewhere in the document. It is generally desirable that the promoter-less marker be located near one terminus of the transposon, and that the 5′ end of the marker be closest to the nearest transposon terminus.

In some embodiments, it may be desirable to identify where a particular transposon has inserted. This can be performed at the level of rough genetic mapping using RFLP-type procedures (restriction followed by size separation of genomic fragments), or by sequencing. The latter permits the precise identification of sequences that have been interrupted, and in some cases, attribute previously undescribed functions to a gene of interest. Sequencing may be effected in a variety of fashions using transposon sequences to prime synthesis into adjacent genomic sequences.

The skilled artisan will also recognize, based on the disclosure set forth herein, that a multitude of organisms, techniques, and metabolic pathways are available for use in the present invention, and that various fermentation products can be obtained as desired according to the present invention.

### EXPERIMENTAL EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

### Experimental Example 1

### Genetic Modification

### Strategies for Ethanol Production

The maximal cell density achieved by growth of Zymomonas mobilis is affected by Type 1 quorum sensing, which utilizes AHL signal molecules. Heterologous expression in Z. mobilis of an AHL-degrading enzyme, such as the PvdQ from P. aeruginosa (nucleic acid sequence, SEQ ID NO:3, and amino acid sequence, SEQ ID NO:4) or AiiA from B. cereus (nucleic acid sequence, SEQ ID NO:5, and amino acid sequence, SEQ ID NO:6), can be used to create a derivative Z. mobilis strain that grows to higher cell density. However, any AHL-acylase or AHL-lactonase that alters or degrades AHL substrates could be used for the same purpose. A Z. mobilis strain that grows to higher density is commercially valuable, for example, for fermentative production of ethanol from sugar, because the volumetric productivity of the reactor increases in direct proportion to the cell density of the fermenting organism.

### Identification of LuxR-Type Proteins in Z. Mobilis.

A LuxR homologue produced by Z. mobilis was identified by amino acid sequence and structural similarity to known LuxR-type proteins. The amino-acid sequence of various known LuxR-type proteins from different organisms, including LuxR from Vibrio fisherii, TraR from A. tumefaciens and SdiA from Escherichia coli, were used as a query to search the Z. mobilis ZM4 genome (Seo et al., 2004, Nat Biotechnol., 23:63-68; GenBank Accession #AE008692) for proteins that have homology to the LuxR protein family. The search was performed using the Psi-BLAST algorithm with default parameters, which calculates a position-specific scoring matrix to identify potentially related sequences. In this example, one predicted protein coding sequence from the ZM4 genome, YP_{—}162698 (nucleic acid sequence, SEQ ID NO:1, and amino acid sequence, SEQ ID NO:2), was uniquely identified with significant homology to LuxR, indicating a probable functional relationship. The identified protein showed a Psi-BLAST Expect score of 4e-06, and was 22% identical (45 of 196 residues) and 46% similar (91 of 196 residues) to LuxR over its length.

### Modeling of the Identified Z. Mobilis LuxR-Type Proteins.

In order to predict the amino acid residues involved in DNA and AHL binding, a structural model of the Z. mobilis LuxR-type protein, YP_{—}162698, was constructed showing its interactions with DNA and with a bound AHL signaling molecule. Homology modeling was used to build a three-dimensional atomic model for YP_{—}162698. To build the model, the YP_{—}162698 amino-acid sequence was aligned with each of the amino-acid sequences of the two existing LuxR-type protein structural templates, TraR from A. tumefaciens and SdiA from Escherichia coli. The alignment was built using several structure-based fold-recognition algorithms using their default parameters, including mGenTHREADER (McGuffin et al., 2003, Bioinformatics, 19:874-881; available at http://bioinfcs.ucl.ac.uk/psipred/) each of which are capable of aligning related protein sequences from distantly related organisms. Model-building software available through the Swiss-Model server was used to build a full-atom homology model for each Z. mobilis protein that is based on the published atomic coordinates for each template structure. The models were validated by examining the protein structure with PROCHECK software (Deptment of Biochemistry & Molecular Biology, University College London, London, GB available at http://www.biochem.ucl.ac.uk/˜roman/procheck/procheck.html), which performs a stereo-chemical analysis of amino-acid residue geometries, and PROVE software (Service de Conformation des Macromolecules Biologiques et de Bioinformatique, Université Libre de Bruxelles, Brussells available at http://www.ucmb.ulb.ac.be/SCMBB/PROVE/), which examines deviation from standard atomic volumes. PROCHECK and PROVE were used with their default parameters to verify that regions of particular interest in the model (e.g. the AHL and DNA binding surfaces) did not deviate significantly from common protein structure. For example, the range of phi and psi angles for peptide bonds was examined with a Ramachandran plot.

The model is used to predict which amino-acid residues in the Z. mobilis homologue protein were likely to be involved in DNA binding. Potential DNA binding contacts in the LuxR homologues from Z. mobilis are identified by inspecting the models for residues in proximity to the bound DNA molecule. A total of 15 residues are identified in YP_{—}162698 that might affect DNA binding directly. Other residues are likely important for AHL binding indirectly, for example, by stabilizing the conformation of other residues that interact directly with the AHL molecule. The model is also used to predict which amino-acid residues in the Z. mobilis homologue protein were likely to be involved AHL binding.

Potential AHL binding contacts in the LuxR homologues from Z. mobilis were identified by inspecting the models for residues in proximity to the bound AHL molecule. A total of 21 residues were identified in YP_{—}162698 that might affect AHL binding directly. Other residues are likely important for DNA binding indirectly, for example, by stabilizing the conformation of other residues that interact directly with the DNA molecule.

### Construction of Mutant LuxR-Type Proteins.

Mutations are introduced into the DNA sequences coding for the Z. mobilis LuxR-type proteins (e.g., YP_{—}162698) that were predicted to do at least one of the following:

1) reduce the affinity of binding between the protein and AHL molecule,

2) destabilize DNA binding at the target DNA activation sequence,

3) destabilize the active conformation of the protein folding switch. Mutations are introduced into the DNA sequences coding for the Z. mobilis LuxR-type proteins (e.g., YP_{—}162698) using both random and site-directed mutagenesis strategies.

To alter particular amino acids that participate in the proper functioning of the protein folding switch, random mutations are introduced throughout the sequence coding for the LuxR-type protein, and are then screened to identify those that can interfere with the protein folding switch mechanism. Random mutagenesis can be performed using the GeneMorph kit (Stratagene, La Jolla, Calif.), or any other method known or predicted to generate random mutations as desired according to the present invention.

Site-directed mutagenesis is performed using the Quick-Site kit (Stratagene, La Jolla, Calif.), or any other suitable kit or method for introducing mutations into a cDNA, to make desired changes to particular amino-acids (e.g., 36 amino acid residues in YP_{—}162698) identified in the homology models and predicted to be important for binding with the DNA molecule, or as important to binding with the AHL molecule. At each site, two different mutations can be introduced to create both a conservative and a non-conservative substitution.

Mutations are introduced into the DNA sequences coding for the Z. mobilis LuxR-type proteins using a combination of the random and site-directed mutagenesis strategies to make combinations of modifications that were predicted to do at least two of the following:

1) reduce the affinity of binding between the protein and AHL molecule,

2) destabilize DNA binding at the target DNA activation sequence, and

3) destabilize the active conformation of the protein folding switch.

Expression of Mutant LuxR-Type Proteins in Z. Mobilis.

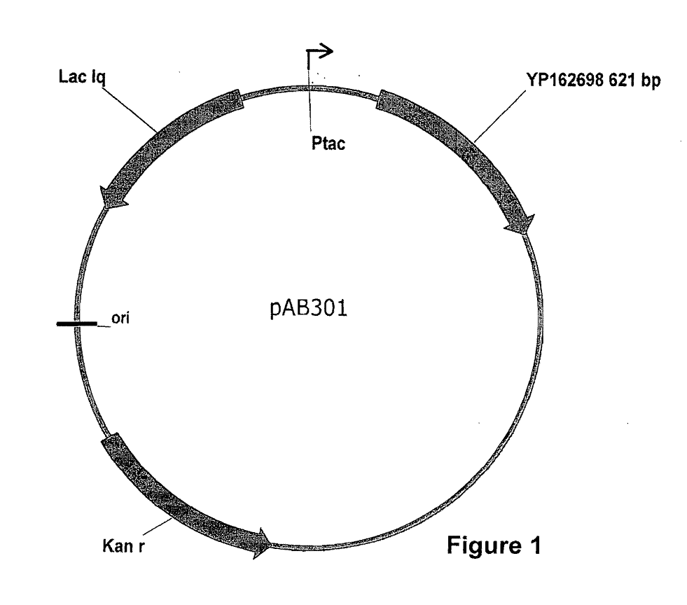

A plasmid expression vector for use in Z. mobilis is constructed according to previously described methods (see generally, Sambrook et al., 2001, Molecular Cloning: A Laboratory Manual, Third Edition, Cold Spring Harbor Laboratory, New York; Ausubel et al., 1997, Current Protocols in Molecular Biology, John Wiley & Sons, New York, and in Gerhardt et al., eds., 1994, Methods for General and Molecular Bacteriology, American Society for Microbiology, Washington, D.C.). In one embodiment of the invention, a DNA fragment containing the E. coli tac promoter sequence (a fusion of the trp and lac promoters) and the constitutively expressed lacI^{Q} repressor gene are amplified by PCR and cloned into the broad host range vector pBBR1MCS-2 (Genbank accession #U23751; Kovach et al., 1994, Biotechniques 16:800-802.) to create plasmid pAB300. The tac promoter functions in Z. mobilis and allows for inducible, differential expression of downstream genes in response to the concentration of IPTG inducer molecule added to the growth media (ref). Other inducible promoter sequences can also be employed for differential expression and based on the disclosure set forth herein, would be understood by those skilled in the art. The LuxR-type protein from Z. mobilis, YP_{—}162698, is then amplified by PCR from ZM4 genomic DNA and cloned downstream from the tac promoter to create plasmid pAB301 (FIG. 1). Clones are constructed in E. coli, and subsequently transferred into Z. mobilis by electroporation using a 10 ms pulse of 12 kV/cm. Transformants are selected and isolated on rich medium (RM) agar plates containing 200 μg ml⁻¹ kanamycin.

The effect of the expressing mutant LuxR-type protein, YP162698, is examined by transforming a Z. mobilis strain with different plasmid expression vectors containing different gene modifications and inducing expression of the cloned gene with IPTG. Particular mutations that affect population cell density were identified by measuring optical density at 600 nm of cultures grown in 1 mL of liquid RM media at 30° C. for 12 hours.

### Inactivation of the Chromosomal Copies of the LuxR-Type Genes.

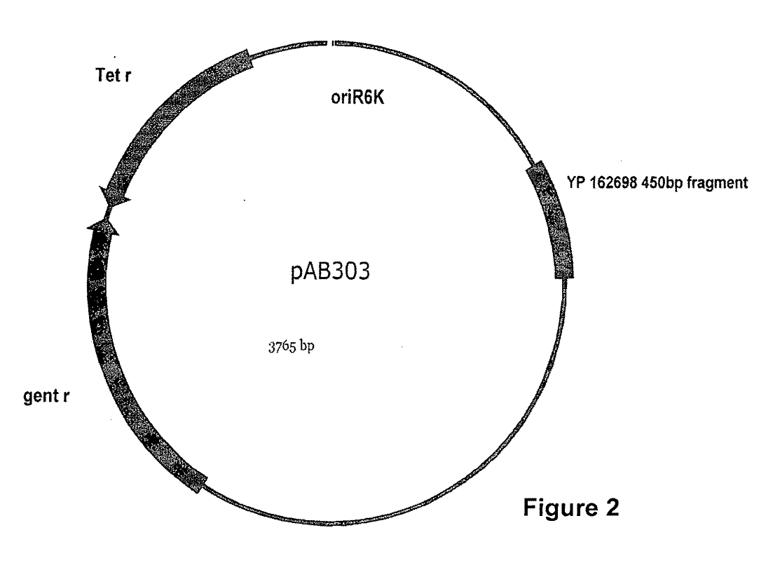

In an embodiment of the invention, to evaluate the effect of expression of the mutant LuxR-type proteins without interference from the existing chromosomal copy of the identified LuxR genes, the chromosomal copy of YP_{—}162698 is inactivated through integration of a suicide vector. The suicide vector e.g., pGPG8, which contains the conditional origin of replication from plasmid R6K (Kolter et al., 1978, Cell 15:1199-1208), replicates in only if Pi protein is present due to expression of the pir gene elsewhere in the cell. Because the pir gene is not carried on the plasmid, the plasmid is unable to replicate, in E. coli or Z. mobilis for example, if Pi protein is absent. A derivative of pGPG8, named pAB302, is constructed by cloning a gene encoding tetracycline resistance downstream of the gene encoding gentamycin resistance. All suicide vectors derived from pGPG8 are propagated in a Pi+ strain of E. coli. Plasmid pAB303 (FIG. 2) is constructed by cloning a 450 by fragment of the YP_{—}162698 gene (including residue 50 to 200 of the predicted coding sequence) into pAB302. This construct is then transformed into Z. mobilis via electroporation. Upon transfer of the suicide vectors into Z. mobilis, the antibiotic resistance gene on the plasmid can only be inherited stably if the plasmid integrates into the chromosome via homologous recombination between the disrupted portion of YP_{—}162698 on the plasmid and the full, active copy of YP_{—}162698 in the chromosome. The result of this integration event is that two disrupted, nonfunctional copies of the YP_{—}162698 gene are created in the chromosome, each flanking the integrated suicide plasmid DNA. The integrated plasmid is stable as long as the contained antibiotic resistance (tetracycline) is selected.

### Experimental Example 2

### Exogenous Agents

### Cloning and Expression of AHL-Degrading Enzymes in Z. Mobilis

Two AHL-degrading enzymes, representing each of the known enzymatic AHL-degrading mechanisms, are expressed in Z. mobilis to disrupt quorum sensing and allow the organism to grow to higher cell density. The enzymes chosen are the AiiA AHL-lactonase from B. cereus, and the PvdQ AHL-acylase from P. aeruginosa.

The broad-host range plasmid described above, pAB300, is used to express both enzymes. The genes encoding AiiA and PvdQ are cloned into pAB300 downstream of the tac promoter using standard molecular biology techniques, thereby creating plasmids pAB310 and pAB320, respectively. Clones are constructed in E. coli, and subsequently transferred into Z. mobilis by electroporation. Transformants are selected and isolated on rich medium (RM) agar plates containing 200 μg ml⁻¹ kanamycin.

The effect of expressing each AHL-degrading enzyme is examined by measuring optical density at 600 nm of cultures grown in 1 L of liquid RM media at 30° C. for 12 hours with varying concentrations of the IPTG inducer added to the media. Increasing concentration of IPTG is correlated with increased final cell density.

### Utilization of Esterase to Degrade Homoserine Lactone (Type 1) Autoinducer Signals.

Four vessels are prepared, each containing 1 liter of liquid RM medium consisting of 2 grams glucose, 1 gram yeast extract (Oxoid L21), and 0.2 grams KH₂PO₄ per 100 milliliters of water.

In two of the vessels, the medium is supplemented with 200 U/ml of the filter sterilized esterase, Sigma #E0887 (Sigma-Aldrich Corp. St. Louis, Mo.), and the medium in the other two vessels is not. Unmodified Z. mobilis bacteria are added to one of the vessels containing esterase and one of the vessels not containing esterase and genetically modified Z. mobilis are added to the other of the vessels containing esterase and the other of the vessels not containing esterase. All four vessels are placed in an incubator at 30° C. for 12 hours.

Cell densities of each of the four cultures is determined by plating serial dilutions of each culture onto RM agar plates, incubating the plates at 30° C. for 12 hours, and counting the colony forming units (CFU). The number of CFU on plates from cultures of genetically modified bacteria should be greater than those on plates containing unmodified bacteria. The number of CFU on the plates from cultures containing esterase should be greater than those on the plates from cultures without esterase.

### Utilization of Solid-Phase-Bound Antibodies to Inhibit a Peptide-Regulated Signaling System.

Antibodies to the autoinducer peptide are generated using methods known in the art. The antibodies are then bound to NHS-activated Sepharose (Amersham Pharmacia Biotech) via primary amino groups according to procedures developed by the manufacturer. A column is prepared containing the antibody-bound Sepharose. A medium is prepared based on the requirements of the microorganism to be used. The sample is added to the Sepharose column and organisms are allowed to bind. The medium is then continuously flushed over the column. In this manner, the autoinducer will be removed by the antibody.

### Utilization of a Continuous Flow-Device to Inhibit Quorum Sensing.

Microorganisms are added to a continuous flow reactor containing liquid RM growth medium, or any other suitable growth medium as described herein. Continuous removal of medium and replacement of removed medium prevents autoinducer levels from reaching threshold levels. Additionally, modulation of other culture conditions (e.g., pH, ionic strength, etc.) and nutrient levels can be used to control autoinducer levels. Fermentation products can be harvested from the removed medium, or from the continuous culture vessel.

### Experimental Example 3

### Screening for “Knockout” Mutations Conferring Growth Effects

The following experiments were designed to screen for genes responsible for production or detection of the quorum sensing signal. It is believed that inactivation of quorum sensing systems does not result in lethal effects.

Random mutagenesis is a preferred strategy for numerous reasons. It is believed that recovered mutations will disrupt enzymes involved in quorum sensing; however, any genetic change conferring increased growth can be recovered. Disruption of genes in non-quorum sensing systems may also provide growth advantages, and identification of these systems may not be predictable by other means. Random mutagenesis is expected to disrupt all genes in the chromosome and should thus identify any useful gene that would be predicted by sequence analysis. In addition, mutagenesis (e.g., insertion mutagenesis) can identify regulatory DNA elements, thus providing information from which genetic engineering strategies can later benefit.

### Methods

Mini-Mu DNA Fragment Construction

Mini-Mu DNA can be prepared using manufactures protocol from pEntransposon-Kan^{R} (Finnzymes, Espoo, Finland). Briefly, pEntransposon-Kan^{R} is digested with BglII to yield “precut” transposon ends having appropriate 4-base 5′ overhangs. This end structure ensures efficient assembly of stable mini-Mu transpososomes, and increases the efficiency of MuA-catalyzed integration over DNA produced directly by PCR.

Transpososome Assembly

Transpososomes can be assembled per manufacture's recommendation (Finnzymes, Espoo, Finland). Briefly, a purified mini-Mu DNA fragment is combined with Mu transposase in a 1:5 molar ratio in 150 mM of Tris-Ha (pH 6.0), 50% (v/v) glycerol, 0.025% (w/v) Triton X-100, 150 mM NaCl, and 0.1 mM EDTA, and incubated at 30° C. for 2 hours. Non-covalent protein-DNA complexes can be detected after electrophoresis on 2% agarose gels at 4° C. Stable transpososomes are visible as bands with decreased mobility and sensitivity to sodium dodecyl sulfate (SDS).

### Growth and Electroporation of Z. Mobilis.

Strain ZM4 was obtained from the ATCC (#31821), are propagated on RM medium (glucose, 20 g L⁻¹; yeast extract, 10 g L⁻¹, KH₂PO₄, 2 g g L⁻¹ (50 mM); pH 6.0) at 30° C. in shake flasks with aeration. ZM4 are electroporated using standard techniques. To prepare electrocompetent cells, ZM4 are grown to OD600 of 0.3-0.4. Cells are harvested by centrifugation, washed twice in ice-cold water, once in 10% glycerol, and the cell pellet is suspended in 10% glycerol. For electroporation, the transpososome mixture is be diluted (1:4 or more) with water, and aliquots containing 20-200 ng of DNA are combined with 25 μl electrocompetent cells in a 1 mm gap cuvette. Electroporation is performed using a Bio-Rad Genepulser: 25 uF; 1.8 kV; 200 Ω. Electroporated cells are incubated in 1 ml of RM medium at 30° C. without shaking to allow phenotypic expression of kanamycin resistance, and then spread on RM media with agar (15 g L⁻¹) and kanamycin (50 ug mL-1) to select for cells containing integrated mini-Mu transposons.

Verification of Inserted Transposons

At least 20 independently isolated colonies can be validated by DNA sequencing to ensure that (1) transposition is occurring, and (2) insertions into unique sites are being recovered. DNA sequencing primers are deigned to both ends of the transposon, and sequencing reactions are performed with chromosomal DNA prepared from each of the 20 mutant strains. The resulting sequencing data are compared to the ZM4 genome sequence to identify the location of each mini-Mu insertion.

Library Construction

Once the mutagenesis procedure has been validated, a library of at least 6000 independent mutants are isolated. Because ZM4 contains approximately 2000 genes, a library of 6000 mutants provides 95% confidence that mutations are recovered in all genes. Each colony are inoculated into one well of a 96-well microtiter plate containing RM media and kanamycin (50 ug mL⁻¹). Plates are incubated to saturation density with aeration at 30° C. For storage, replica plates containing glycerol can be stored at −80° C.

### Transposon Insertion Mutagenesis

Mutagenesis is a powerful approach for studying the genes involved in complex microbial processes. When the genes have yet to be elucidated, an efficient strategy for their identification is to create a library of “knockout” mutations in all possible nonessential genes. The resulting library can then be tested for phenotypes of interest. Determining the location of the knockout mutation provides the identity of the disrupted gene or regulatory sequence (e.g. promoter, operator).

Transposon insertion mutagenesis strategies provide an efficient means to construct random insertion libraries. Transposon mutagenesis usually creates stable, polar mutations that completely inactivate the gene into which the transposon has inserted. The location of the insertion can be readily determined through DNA sequencing initiated from the known sequences of the transposon ends. Transposon mutagenesis has been successfully demonstrated in Z. mobilis using “mini-Mu”, which is a derivative of bacteriophage Mu. Mu replicates its genome though transposition and is one of the best studies mobile elements. However, an active Mu element in the chromosome is not stable because it will continue to replicate its genome through transposition to other sites in the chromosome. Two strategies exist for preventing subsequent Mu transposition after a desirable initial event.

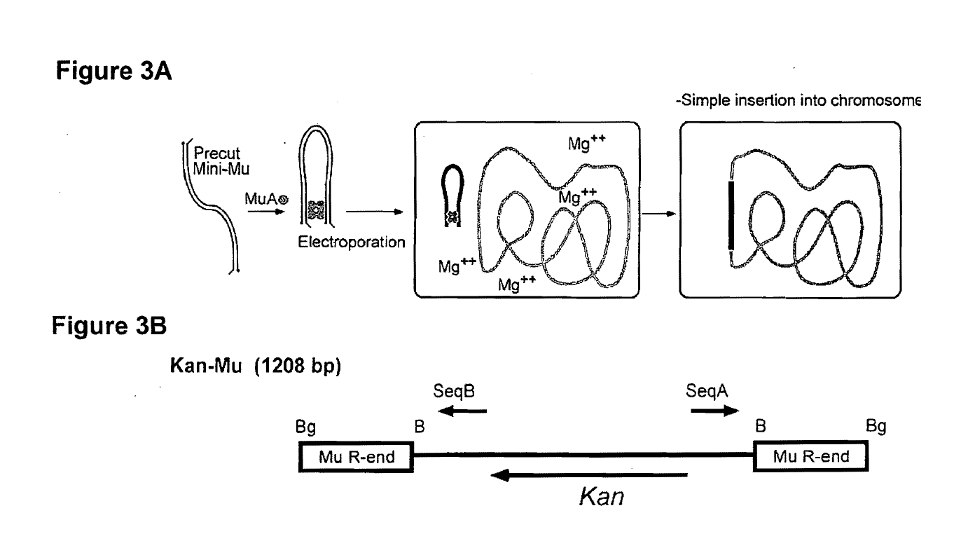

The first strategy uses a recently developed two-step approach in which a “transpososome” is first assembled in vitro from purified MuA transposase and a synthetic DNA sequence. The mini- Mu DNA molecule contains binding sites for transposase at both ends flanking an antibiotic resistance gene (FIG. 3). The stable transpososome complex is introduced into cells via electroporation. Upon entering the cell, the transpososome is activated by magnesium ions and catalyzes random insertion of the synthetic DNA sequence into the chromosome.

The second strategy utilizes related Mu derivatives (termed Mud) lacking the MuA transposase gene. Transposition is enabled by expressing MuA transposase from a gene outside of the Mud element, termed “transitory cis-complementation”. Transitory cis-complementation allows the Mud to hop randomly into the chromosome; however, further transposition is prevented because the gene encoding MuA is left behind. Typically, the delivered DNA carrying Mud and MuA is a non-replicating element (e.g. a conjugated “suicide” plasmid or DNA transduced by a bacteriophage), and the MuA gene is rapidly and permanently lost from the cell.

In vitro assembly and electroporation of transpososomes is a simple and efficient approach to creating an insertion library. It has been successful in many gram-negative bacterial species, including Salmonella typhimurium LT2, Erwinia carotovora, and Yersinia enterocolitica. The MuA transposase is known to be active in Z. mobilis and therefore would be applicable to Z. mobilis.

In E. coli, the combined efficiency of electroporation and transpososome integration was 1000-fold reduced from the efficiency of electroporation. High-efficiency electroporation of Z. mobilis achieving up to 107 transformants per ug DNA has been reported; the efficiency of transpososome electroporation and integration is therefore expected to approach 104 transformants per ug DNA, which is sufficient to construct the required library of 6000 strains.

Z. mobilis strain Zm4 is known to transform 10- to 1000-fold more efficiently with unmethylated DNA over DNA methylated by E. coli. If necessary to improve the efficiency of transposon integration, DNA with appropriate methylation can be prepared prior to BglII digestion by: (1) purification from a methylation-deficient E. coli strain such as JM110 (ecoK-) or HB101 (damdcm-), (2) purification from Z. mobilis ZM4 directly, or (3) synthesis by PCR.

MuA transposase is commercially available from Finnzymes (Espoo, Finland) and Epicentre (Madison Wis.). Epicentre offers a derivative of the MuA transposase known as HyperMu™ that is 50-times more active than the wild-type enzyme in vitro and retains its random insertion specificity. Use of HyperMu transposase is preferred because it may increase the frequency of insertion, thereby increasing the overall efficiency of library construction.

An alternative strategy is to deliver the “mini-Mu” by plasmid conjugation. Although more complex and time consuming due to the additional molecular-biology work required to create the necessary DNA constructs, this approach is known to work in Z. mobilis. Briefly, derivatives of conjugal plasmids such as RP4 are known to mate between bacterial species, and conjugal transfer of plasmid DNA between E. coli and Z. mobilis has been demonstrated. Standard molecular biology techniques can be used to construct a derivative of an appropriate conjugative plasmid containing the kanamycin-resistant mini-Mu and MuA transposase gene. Mini-Mu can be amplified by PCR from the pEntransposon-KanR plasmid (Epicentre). The gene encoding MuA can be amplified by PCR from an E. coli strain infected with the Mu bacteriophage. Matings between E. coli and Z. mobilis are performed essentially as described elsewhere herein by using filter paper on RM media to provide an appropriate surface. After plasmid DNA is transferred to Z. mobilis and ransposition has occurred, the mixed strains are resuspended in liquid RM media and incubated for several hours to allow phenotypic expression of kanamycin resistance. The chosen conjugative plasmid are unable to replicate in Z. mobilis; therefore, kanamycin resistance will only be stably inherited through transposition of mini-Mu into the chromosome. The mixed bacterial culture are then spread on solid RM media supplemented with kanamycin to select for ZM4 derivatives containing the integrated transposon. A counterselection against E. coli cells can be performed by including a second antibiotic to which only Z. mobilis is resistant. Strain ZM4 is naturally resistant to many antibiotics that can be used.

Another strategy involves a targeted strategy to disrupt the identified gene directly in order to disrupt quorum sensing. Briefly, a nonreplicating “suicide” plasmid is constructed containing an inactive, truncated fragment of the identified target gene and a selectable antibiotic resistance marker. When the suicide-vector is electroporated into Z. mobilis, it can only be stably inherited if homologous recombination occurs between the active gene in the chromosome and the inactive copy on the plasmid, thereby inactivating the chromosomal gene. Integration events are selected using the antibiotic resistance gene carried on the suicide plasmid.

### Screening

To screen the entire knockout library for insertion mutations, cultures are grown in microtiter plates and a multi-well spectrophotometer is used to measure the optical density of all cultures on each plate simultaneously. Optical density is measured continuously to allow both effects to be identified. Changes in growth rate is also monitored. It is believed that overgrowth will manifest as at least a 10% increase in optical density. The screening method would encompass inoculating each of the mutant strains into RM media in microtiter plates and incubated at 30° C. in an orbital shaker to saturation cell density. Multiple plates can be handled simultaneously. Wild-type ZM4 is inoculated into several positions on each plate for controls. Using a multi-channel pipettor, saturated cultures are inoculated with a 1:10 dilution into 200 ul of fresh RM media in microtiter plates. Plates are incubated with aeration at 30° C. A fraction of the wild-type cultures are supplemented with enzyme as positive controls; other wild-type cultures are incubated without enzyme as negative controls. OD measurements are directly recorded every hour for the first eight hours to identify cultures displaying a reduced lag phase duration, and establish a rate for logarithmic growth. After 24 hours, optical density is recorded at stationary phase. To guard against inaccurate readings at high densities (OD greater than 2.0-3.0), saturated cultures are diluted 1:10 into RM media prior to measurement.

In order to validate the mutant strains, cultures displaying enhanced growth are selected for “follow-up” testing. Enhanced growth is defined as: (1) reduced lag phase duration, (2) increased logarithmic growth rate, or (3) increased density at stationary phase. To limit the number of strains for follow-up, only mutants showing improvements greater than 10% are examined further. Selected mutants are re-tested to confirm their phenotype(s) and measure the magnitude of each enhancement. To improve accuracy, cultures are tested in replicate, and increased culture volumes are used.

Further characterization of the mutant strain can provide further insight to their potential utility and mechanism of action. First, DNA sequencing is performed to identify the location of each insertion mutagenisis. Second, the sensitivity of each mutant to media conditioned by wild-type ZM4 is examined. It is believed that the mutations might act by disrupting either the production or detection of QS signals by Z. mobilis. Disruption of QS signal detection is expected to render the mutant strain resistant to exogenously added QS signals. In contrast, disruption of QS signal production is likely to create a mutant strain that can still be inhibited by exogenous signals. Third, the dry cell mass and total amount of cellular protein per unit culture volume is determined. Dry cell mass correlates with the total amount of enzyme catalyst produced in the culture, and is expected to increase in proportion to the cultures overall biosynthetic potential for ethanol production. Fourth, to estimate the ability of each mutant to produce ethanol with enhanced volumetric productivity, the volumetric activity of the ADH gene is measured in a crude cell extract. Alcohol dehydrogenase (ADH) is a key enzyme in the production of ethanol by Z. mobilis, and increased ADH activity per unit culture volume is expected to correlate with enhanced fermentation productivity.

A collection of mutations and their associated effects can be tabulated. The most promising strains can be identified for further work. It is expected that these strains will compose a collection differentially acting mechanisms that can be combined for additive or synergistic benefits.

The disclosures of each and every patent, patent application, and publication cited herein are hereby incorporated herein by reference in their entirety.

While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

What is claimed:

1. A genetically modified known microorganism comprising at least one genetic mutation, wherein said mutation confers upon said genetically modified microorganism the ability to grow to a greater cell density than the cell density of an otherwise identical microorganism that does not comprise said mutation and is cultured under identical culture conditions.

2. The genetically modified microorganism of claim 1, wherein the mutation is within the regulatory region of a gene associated with quorum sensing.

3. The genetically modified microorganism of claim 1, wherein the mutation is in a nucleic acid sequence encoding a quorum sensing protein; wherein said mutation modulates at least one of:
a. the production of said quorum sensing protein;
b. the half-life of said quorum sensing protein;
c. the response of said quorum sensing protein to a quorum sensing signal;
d. the activity of said quorum sensing protein; and
e. the interaction of said quorum sensing protein with a quorum sensing pathway in said microorganism.

4. The genetically modified microorganism of claim 3, wherein said mutation modulates production and/or activity of at least one polypeptide involved in quorum sensing signaling in at least one pathway selected from the group consisting of a Type 1 quorum sensing pathway, a Type 2 quorum sensing pathway, and a peptide-mediated quorum sensing pathway.

5. The genetically modified microorganism of claim 3, wherein said mutation is a transposable interruptor resulting in interruption of the nucleic acid encoding a polypeptide involved in quorum sensing signaling in at least one pathway selected from the group consisting of a Type 1 quorum sensing pathway, a Type 2 quorum sensing pathway, and a peptide-mediated quorum sensing pathway.

6. The genetically modified microorganism of claim 3, wherein said mutation is in a nucleic acid sequence encoding a LuxR-type protein; wherein said mutation modulates at least one of:
a. the binding of said LuxR-type protein to DNA;
b. the binding of said LuxR-type protein to an acyl homoserine lactone (AHL); and
c. the protein folding switch of said LuxR-type protein. conditions.

7. A genetically modified known microorganism comprising at least one genetic, wherein said mutation confers upon said genetically modified microorganism the ability to achieve a higher volumetric productivity for a fermentation product produced by said microorganism than the volumetric productivity for the same fermentation product by an otherwise identical microorganism that does not comprise said mutation.

8. The genetically modified microorganism of claim 7, wherein the mutation is within the regulatory region of a gene associated with quorum sensing.

9. The genetically modified microorganism of claim 7, wherein the mutation is in the nucleic acid sequence encoding a quorum sensing protein; wherein said mutation modulates at least one of:
a. the production of said quorum sensing protein;
b. the half-life of said quorum sensing protein;
c. the response of said quorum sensing protein to a quorum sensing signal;
d. the activity of said quorum sensing protein; and
e. the interaction of said quorum sensing protein with a quorum sensing pathway in said microorganism.

10. The genetically modified microorganism of claim 9, wherein said mutation modulates production and/or activity of at least one polypeptide involved in quorum sensing signaling in at least one pathway selected from the group consisting of a Type 1 quorum sensing pathway, a Type 2 quorum sensing pathway, and a peptide-mediated quorum sensing pathway.

11. The genetically modified microorganism of claim 9, wherein said mutation is a transposable interruptor resulting in interruption of the nucleic acid encoding a polypeptide involved in quorum sensing signaling in at least one pathway selected from the group consisting of a Type 1 quorum sensing pathway, a Type 2 quorum sensing pathway, and a peptide-mediated quorum sensing pathway.

12. The genetically modified microorganism of claim 9, wherein said mutation is in a nucleic acid sequence encoding a LuxR-type protein; wherein said mutation modulates at least one of:
a. the binding of said LuxR-type protein to DNA;
b. the binding of said LuxR-type protein to an acyl homoserine lactone (AHL); and
c. the protein folding switch of said LuxR-type protein. conditions.

13. A method of increasing the cell density of a population of known microorganisms, said method comprising:
a. introducing a genetic modification into a microorganism; and
b. growing the genetically modified microorganism in a culture medium,
whereby said modified microorganism grows to a greater cell density than the cell density of an otherwise identical microorganism that does not comprise said mutation and is cultured under identical culture conditions.

14. The method of claim 13, wherein the genetic modification is a mutation within the regulatory region of a gene associated with quorum sensing.

15. The method of claim 13, wherein the genetic modification is a mutation in a nucleic acid sequence encoding a quorum sensing protein; wherein said mutation modulates at least one of:
a. the production of said quorum sensing protein;
b. the half-life of said quorum sensing protein;
c. the response of said quorum sensing protein to a quorum sensing signal;
d. the activity of said quorum sensing protein; and
e. the interaction of said quorum sensing protein with a quorum sensing pathway in said microorganism;.

16. The method of claim 15, wherein the mutation modulates production and/or activity of at least one polypeptide involved in quorum sensing signaling in at least one pathway selected from the group consisting of a Type 1 quorum sensing pathway, a Type 2 quorum sensing pathway, and a peptide-mediated quorum sensing pathway.

17. The method of claim 15, wherein the mutation is a transposable interruptor resulting in interruption of the nucleic acid encoding a polypeptide involved in quorum sensing signaling in at least one pathway selected from the group consisting of a Type 1 quorum sensing pathway, a Type 2 quorum sensing pathway, and a peptide-mediated quorum sensing pathway.

18. The method of claim 15, wherein the mutation is in a nucleic acid sequence encoding a LuxR-type protein; wherein said mutation modulates at least one of:
a. the binding of said LuxR-type protein to DNA;
b. the binding of said LuxR-type protein to an acyl homoserine lactone (AHL); and
c. the protein folding switch of said LuxR-type protein. conditions.

19. A method of increasing the volumetric productivity of a population of known microorganisms, said method comprising:
a. introducing a genetic modification into a microorganism; and
b. growing the modified microorganism in a culture medium,
wherein the volumetric productivity of said modified microorganism with respect to a fermentation product produced by said microorganism is greater than the volumetric productivity for the same fermentation product by an otherwise identical microorganism that does not comprise said mutation.

20. The method of claim 19, wherein the genetic modification is a mutation within the regulatory region of a gene associated with quorum sensing.

21. The method of claim 19, wherein the genetic modification is a mutation in a nucleic acid sequence encoding a quorum sensing protein; wherein said mutation modulates at least one of
a. the production of said quorum sensing protein;
b. the half-life of said quorum sensing protein;
c. the response of said quorum sensing protein to a quorum sensing signal;
d. the activity of said quorum sensing protein; and
e. the interaction of said quorum sensing protein with a quorum sensing pathway in said microorganism.

22. The method of claim 21, wherein the mutation modulates production and/or activity of at least one polypeptide involved in quorum sensing signaling in at least one pathway selected from the group consisting of a Type 1 quorum sensing pathway, a Type 2 quorum sensing pathway, and a peptide-mediated quorum sensing pathway.

23. The method of claim 21, wherein the mutation is a transposable interruptor resulting in interruption of the nucleic acid encoding a polypeptide involved in quorum sensing signaling in at least one pathway selected from the group consisting of a Type 1 quorum sensing pathway, a Type 2 quorum sensing pathway, and a peptide-mediated quorum sensing pathway.

24. The method of claim 21, wherein the mutation is in a nucleic acid sequence encoding a LuxR-type protein; wherein said mutation modulates at least one of:
a. the binding of said LuxR-type protein to DNA;
b. the binding of said LuxR-type protein to an acyl homoserine lactone (AHL); and
c. the protein folding switch of said LuxR-type protein. conditions.

25. A method of increasing the cell density of a population of known microogranism, said method comprising:
a. introducing into a microorganism a nucleic acid vector comprising a nucleic acid sequence encoding a polypeptide, wherein the polypeptide has the ability to modulate at least one quorum sensing pathway;
b. expressing said polypeptide within said microorganism; and
c. growing the modified microorganism in a culture medium
whereby said modified microorganism grows to a greater cell density than the cell density of an otherwise identical microorganism that does not comprise said polypeptide and is cultured under identical culture conditions.

26. A method of increasing the volumetric productivity of a population of known microorganism, said method comprising:
a. introducing into a microorganism a nucleic acid vector comprising a nucleic acid sequence encoding a polypeptide, wherein the polypeptide has the ability to modulate at least one quorum sensing pathway;
b. expressing said polypeptide within said microorganism; and
c. growing the modified microorganism in a culture medium,
wherein the volumetric productivity of said modified microorganism with respect to a fermentation product produced by said microorganism is greater than the volumetric productivity for the same fermentation product by an otherwise identical microorganism that does not comprise said polypeptide.

27. A method of producing a fermentation product, said method comprising:
a. providing a genetically modified known microorganism comprising at least one mutation in a nucleic acid sequence encoding a quorum sensing protein; wherein said mutation modulates at least one of:
i. the production of said quorum sensing protein;
ii. the half-life of said quorum sensing protein;
iii. the response of said quorum sensing protein to a quorum sensing signal;
iv. the activity of said quorum sensing protein; and
v. the interaction of said quorum sensing protein with a quorum sensing pathway in said microorganism; and
b. culturing said genetically modified microorganism in a culture medium;
wherein said mutation confers upon said genetically modified microorganism the ability to achieve a higher volumetric productivity for a fermentation product produced by said microorganism than the volumetric productivity for the same fermentation product by an otherwise identical microorganism that does not comprise said mutation.

28. A method for the production of a fermentation product according to claim 27, further comprising harvesting at least one fermentation product from the culture medium.

29. The method of claim 27, wherein said fermentation product is at least one fermentation product selected from the group consisting of: lactate, acetate, succinate, formate, butyrate, ethanol, butanol, acetone, and butanediol.

30. The method of claim 27, wherein said fermentation product is ethanol.

31. A method of producing a fermentation product, said method comprising:
a. introducing into a known microorganism a nucleic acid vector comprising a nucleic acid sequence encoding a polypeptide, wherein the polypeptide has the ability to modulate at least one quorum sensing pathway; and
b. culturing said genetically modified microorganism in a culture medium;
wherein said modified microorganism has the ability to achieve a higher volumetric productivity for a fermentation product produced by said microorganism than the volumetric productivity for the same fermentation product by an otherwise identical microorganism that does not comprise said polypeptide.

32. A method of identifying a gene associated with quorum sensing, wherein mutation of said gene in a microbial cell allows the cell to grow at an increased density, the method comprising:
a. introducing a library of mutant nucleic acid fragments into a plurality of cells;
b. selecting a cell exhibiting increased cell growth;
c. isolating the mutated nucleic acid sequence from said cell exhibiting increased cell growth;
d. sequencing the mutated nucleic acid;
e. analyzing the sequence of the mutated nucleic acid sequence;
thereby identifying a gene associated with quorum sensing.
